(19) United States
(12) Patent Application Publication
(10) Pub. No.: US 2005/0186276 A1
(43) Pub. Date: Aug. 25, 2005

(54) Pharmaceutical formulations

(75) Inventors: Alfred Berchielli, Niantic, CT (US); Avinash G. Thombre, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/878,907

(22) Filed: Jun. 28, 2004

Related U.S. Application Data
(60) Provisional application No. 60/488,229, filed on Jul. 17, 2003.

(51) Int. Cl. A61K 9/22; A61K 31/455; A61K 31/4433

(52) U.S. Cl. 424/468; 514/337; 514/355

(57) ABSTRACT

The present invention provides controlled-release pharmaceutical formulations comprising phosphodiesterase type 4D (PDE4D) inhibitors having improved release profiles to an environment of use.

### BACKGROUND OF THE INVENTION

The invention relates to controlled-release pharmaceutical formulations providing a controlled-release of a beneficial agent to an environment of use. More specifically, the invention provides controlled-release pharmaceutical formulations comprising phosphodiesterase type 4D (PDE4D) inhibitors.

The PDE4D inhibitors, and the pharmaceutically acceptable salts thereof, useful in the controlled-release formulations of the present invention, will be well known to one of ordinary skill in the art. A number of selective inhibitors of PDE4D have been discovered recently, and beneficial pharmacological effects resulting from that inhibition have been demonstrated in a number of disease models. See, for example, Torphy, et al., Environ. Health Perspect., 102, Suppl. 10, 79-84 (1994); Duplantier, et al., J. Med. Chem., 39, 120-125 (1996); Schneider, et al., Pharmacol. Biochem. Behav., 50, 211-217 (1995); Banner and Page, Br. J. Pharmacol., 114, 93-98 (1995); Barnette, et al., J. Pharmacol. Exp. Ther., 273, 674-679 (1995); Wright, et al., Can. J. Physiol. Pharmacol., 75, 1001-1008 (1997); Manabe, et al., Eur. J. Pharmacol., 332, 97-101 (1997); and Ukita, et al., J. Med. Chem., 42, 1088-1099 (1999). PDE4D inhibitors are known to be useful in the treatment of a number of inflammatory, respiratory, and allergic disorders and conditions mediated by the PDE4D isozyme including, but not limited to, asthma; chronic obstructive pulmonary disease (COPD), including chronic bronchitis, emphysema, and bronchiectasis; chronic rhinitis; and chronic sinusitis. Within the airways of patients suffering from asthma and other obstructive airway disease, PDE4D is the most important of the PDE isozymes as a target for drug discovery because of its ubiquitous distribution in airway smooth muscle and inflammatory cells. Airflow obstruction and airway inflammation are features of asthma as well as COPD. Thus, PDE's such as PDE4D that are involved in smooth muscle relaxation, and are also found in eosinophils as well as neutrophils, are believed to constitute an essential element in the etiology of both diseases.

Although many PDE4D inhibitors introduced to the art have been designed to have reduced gastrointestinal and central nervous system side-effects, nausea and emesis, i.e., vomiting, continue to present in many patients being treated with such inhibitors. The mechanism(s) by which PDE4D inhibitors induce nausea and/or emesis is/are presently unknown, however, it is currently believed that such effects are at least partially mediated by emesis centers in the brain, and/or by local gastrointestinal disturbance.

The controlled-release formulations of the present invention provide improved release profiles of PDE4D inhibitors to an environment of use. Such improved release profiles afford PDE4D formulations allowing once or twice daily dosing regimens, with concomitant significant reduction in both the nausea and emesis induced by the administration of such inhibitors.

### SUMMARY OF THE INVENTION

The present invention provides controlled-release pharmaceutical formulations comprising phosphodiesterase type 4D (PDE4D) inhibitors having improved release profiles to an environment of use.

### BRIEF DESCRIPTION OF THE DRAWINGS

FIG. 1 shows the average release rate of the 10 and 25 mg formulations of HPMC and PEO based matrix tablets.

FIG. 2 shows the average release rate of the 10 mg ESC controlled-release formulations tableted with 5/16″ and 11/32″ SRC tooling.

FIG. 3 shows the average release rate of the 10 mg AMT controlled-release formulations tableted with 5/16″ and 11/32″ SRC tooling.

FIG. 4 shows the average release rate of the 4 formulations of HPMC based matrix tablets. The results indicate that the higher molecular weight polymer (Methocel® K4M) may initially release faster than the lower molecular weight K100 LV CR. After the polymer gels more completely (after 2 hours) the trend is reversed as would be expected. Formulations made with jet-milled (I) exhibited much faster and more complete release within a 24-hour period.

FIG. 5 shows the average release rate of the 5 mg formulations tabletted with 11/32″ SRC and modified oval tooling. The formulations with the jet-milled (Ia) showed much faster and more complete release within 24 hours compared to the formulations that were not jet-milled. The modified oval tooling showed a faster release rate compared to the SRC shape. Release was independent of the amount of coating over the 11 to 14% range.

### DETAILED DESCRIPTION OF THE INVENTION

In a first embodiment, the invention provides controlled-release pharmaceutical formulations comprising a phosphodiesterase type 4D (PDE4D) inhibitor, or a pharmaceutically acceptable salt thereof, which formulations exhibit at least one of the following characteristics:
(i) a Tₘₐₓ of greater than about 1.5 hours;
(ii) less than about 80% of the PDE4D inhibitor, or the pharmaceutically acceptable salt thereof, is released in vivo at about 1.5 hours;
(iii) less than about 80% of the PDE4D inhibitor, or the pharmaceutically acceptable salt thereof, is released in vitro at about 1.5 hours; or
(iv) an in vivo delivery lag time prior to initiation of release of the PDE4D inhibitor, or the pharmaceutically acceptable salt thereof, of between about 0.5 hours and about four hours, wherein the formulations comprise an asymmetric membrane-coated osmotic tablet, capsule, or bead core comprising:
(a) a PDE4D inhibitor, or a pharmaceutically acceptable salt thereof;
(b) one or more osmotic agents; and
(c) at least one asymmetric membrane coating the tablet, capsule, or bead core.

### AMT Formulations

The controlled-release formulations of the first embodiment of the invention utilize so-called “AMT” (asymmetric membrane technology), and may be prepared as disclosed in, for example, U.S. Pat. No. 5,612,059, the disclosure of which is incorporated herein by reference in its entirety, or PCT International Application Publication No. WO 2002/17918. Controlled-release AMT formulations typically comprise an asymmetric membrane-coated osmotic tablet, capsule, or bead core comprising: (a) an active drug substance; (b) one or more osmotic agents; (c) one or more solubilizing agents; and (d) at least one asymmetric membrane coating the tablet, capsule, or bead core.

With respect to all controlled-release formulations of the instant invention, the active drug substance comprises a PDE4D inhibitor, or a pharmaceutically acceptable salt thereof. Preferred PDE4D inhibitors, useful in the practice of the instant invention, are disclosed in detail hereinbelow.

The core includes an osmagent (e.g., an osmotic agent). The osmagent provides the driving force for transport of water from the environment of use into the core of the device. The rate of water transport from the environment of use into the core is dependent on the osmotic pressure generated by the core components and the permeability of the membrane coating. The osmagent is generally present in the core at a concentration from about 20% to about 80% by weight, preferably from about 25% to about 75%, more preferably from about 35% to about 50%, still more preferably from 40% to about 60%. A wide variety of osmagents can provide the osmotic pressure needed to drive the drug from the osmotic device.

The osmagent is selected so that an appropriate osmotic pressure (and drug solubility) is provided in the core that results in achieving the target drug-release rate and target delivery duration. Ideally, the drug/osmagent ratio in the core formulation is equal to the ratio of their respective solubilities. Since the drug release rate also depends on the membrane permeability, the core and coating should be optimized iteratively.

Osmagents are usually solutes with a pH-independent aqueous solubility. Traditionally, sugars and inorganic salts are employed. Preferred osmagents include fructose, lactose, maltose, mannitol, sorbitol, sucrose, xylitol, di-basic and mono-basic potassium phosphate, and potassium chloride, and sodium chloride.

The core formulation may also comprise a solubilizing agent(s) that controls the pH of the core, thereby affecting the solubility of the active drug agent. In most instances, the solubilizing agent is included to increase the solubility of active drug agents that have low aqueous solubility. In some instances, the pH-controlling agent is used to lower the solubility of highly water-soluble active drug agents. The solubilizing agent, by virtue of its effect on the solubility of the active drug agent, also affects the osmotic pressure of the core.

The solubilizing agent is chosen such that the active drug agent has an appropriate solubility in solutions containing the agent. The rate of release of the solubilizing agent may be important and, ideally, the solubilizing agent is chosen such that it remains available in the core essentially over the entire drug delivery period. The solubilizing agent(s) is typically present in the core formulation in amounts ranging up to about 50% w/w of the core. Examples of solubilizing agents include surfactants, pH control agents, such as buffers, organic and inorganic acids, and organic acid salts; organic and inorganic bases; mono-, di-, and tri-glycerides; glyceride derivatives; polyhydric alcohol esters; polyethylene glycol (PEG) and polypropylene glycol (PPG) esters; polyoxyethylene and polyoxypropylene ethers and their copolymers; phospholipids, such as lecithin; sorbitan esters; polyoxyethylene sorbitan esters; carbonate salts; zeolites; and cyclodextrins. Generally preferred surfactants may comprise, for example, lapyrium chloride; laureth 4, i.e., α-dodecyl-ω-hydroxy-poly(oxy-1,2-ethanediyl) or polyethylene glycol monododecyl ether; laureth 9, i.e., a mixture of polyethylene glycol monododecyl ethers averaging about 9 ethylene oxide groups per molecule; monoethanolamine; nonoxynol 4, 9 and 10, i.e., polyethylene glycol mono(p-nonylphenyl) ether; nonoxynol 15, i.e., α-(p-nonylphenyl)-ω-hydroxypenta-deca(oxyethylene); nonoxynol 30, i.e., α-(p-nonylphenyl)-ω-hydroxytriaconta(oxyethylene); poloxalene, i.e., nonionic polymer of the polyethylene-polypropylene glycol type, MW=approx. 3000; polyoxyl 8, 40 and 50 stearate, i.e., poly(oxy-1,2-ethanediyl), α-hydro-ω-hydroxy-; octadecanoate; polyoxyl 10 oleyl ether, i.e., poly(oxy-1,2-ethanediyl), α-[(Z)-9-octadecenyl-ω-hydroxy-; polysorbate 20, i.e., sorbitan, monododecanoate, poly(oxy-1,2-ethanediyl); polysorbate 40, i.e., sorbitan, monohexadecanoate, poly(oxy-1,2-ethanediyl); polysorbate 60, i.e., sorbitan, monooctadecanoate, poly(oxy-1,2-ethanediyl); polysorbate 65, i.e., sorbitan, trioctadecanoate, poly(oxy-1,2-ethanediyl); polysorbate 80, i.e., sorbitan, mono-9-monodecenoate, poly(oxy-1,2-ethanediyl); polysorbate 85, i.e., sorbitan, tri-9-octadecenoate, poly(oxy-1,2-ethanediyl); sodium lauryl sulfate; sorbitan monolaurate; sorbitan monooleate; sorbitan monopalmitate; sorbitan monostearate; sorbitan sesquioleate; sorbitan trioleate; and sorbitan tristearate. The preferred cyclodextrin solubilizers will be well-known to one of ordinary skill in the art, and comprise a family of natural cyclic oligosaccharides capable of forming inclusion complexes with a variety of materials. Preferred cyclodextrins may comprise, for example, those having 6-, 7-, and 8-glucose residues in a ring, commonly referred to as α-cyclodextrins, β-cyclodextrins, and γ-cyclodextrins, respectively. Especially preferred cyclodextrins comprise α-cyclodextrin, β-cyclodextrin, and γ-cyclodextrin, δ-cyclodextrin, and cationized cyclodextrins.

The preferred solubilizer for a specific PDE4D inhibitor is dependent upon the physicochemical properties thereof, such as salt form, intrinsic solubility and pKa, i.e., the pH-dependent solubility of the PDE4D inhibitor, and will be apparent to one skilled in the relevant art. A preferred class of solubilizers for basic PDE4D inhibitors comprises organic acids. A generally preferred subset of organic acids comprises citric, succinic, fumaric, adipic, malic and tartaric acids. Exemplary classes of solubilizers for acidic PDE4D inhibitors comprise alkylating agents, buffering agents, and organic bases. Preferred examples of alkylating or buffering agents include potassium citrate, sodium bicarbonate, sodium citrate, dibasic sodium phosphate, and monobasic sodium phosphate. Examples of organic bases include meglumine, monoethanolamine, diethanolamine, and triethanolamine.

If desired, and/or appropriate, the AMT formulations of the invention may further comprise a concentration-enhancing polymer that enhances the concentration of the PDE4D inhibitor. Suitable concentration-enhancing polymers may comprise, for example, ionizable and non-ionizable cellulosic polymers, such as cellulose esters, cellulose ethers, and cellulose esters/ethers; and vinyl polymers and copolymers having substitutents selected from the group consisting of hydroxyl, alkylacyloxy, and cyclicamido, such as polyvinyl pyrrolidone and polyvinyl acetate. Additionally preferred polymers may comprise, for example, hydroxypropylmethyl cellulose acetate succinate (HPMCAS), hydroxypropylmethyl cellulose (HPMC), hydroxypropylmethyl cellulose phthalate (HPMCP), cellulose acetate phthalate (CAP), and cellulose acetate trimellitate (CAT).

Where appropriate, and or desired, the AMT formulation may further comprise additional conventional excipients such as those that promote performance, tableting, or processing of the formulation. Such excipients include tableting aids, surfactants, diluents, water-soluble polymers, pH modifiers, fillers, binders, pigments/dyes, disintegrants, and lubricants. Exemplary excipients include microcrystalline cellulose; metallic salts of acids, such as aluminum stearate, calcium stearate, magnesium stearate, sodium stearate, and zinc stearate; fatty acids, hydrocarbons and fatty alcohols, such as stearic acid, palmitic acid, liquid paraffin, stearyl alcohol, and palmitol; fatty acid esters, such as glyceryl (mono- and di-) stearates, triglycerides, glyceryl (palmitic stearic) ester, sorbitan monostearate, saccharose monostearate, saccharose monopalmitate, and sodium stearyl fumarate; alkyl sulfates, such as sodium lauryl sulfate and magnesium lauryl sulfate; polymers, such as polyethylene glycols, polyoxyethylene glycols, and polytetrafluoroethylene; and inorganic materials, such as talc and dicalcium phosphate. Either animal or vegetable source magnesium stearate may be employed, however, the vegetable source material is generally preferred.

Methods of manufacturing AMT tablet cores are well-known to those skilled in the relevant art and include, for example, conventional direct compression, dry granulation, wet granulation, and the like. Similarly, AMT capsules can be manufactured by a dip-coating method wherein a stainless steel mandrel is dipped into a solution of the coating polymer. AMT bead cores can be manufactured by methods well known in the relevant art for manufacturing multiparticulates.

The asymmetric membrane coating the tablet or bead core typically comprises cellulose acetate (CA) and PEG. The ratio of CA to PEG influences the permeability of the coating and therefore, affects the rate of drug release. Increasing the PEG content increases the aqueous permeability of the membrane. The coating weight also affects the active drug agent release rate; heavier and/or thicker coatings generally decrease the drug release rate. The CA/PEG ratio and the coating weight are chosen such that the membrane coating has sufficient mechanical strength, the coating process is completed in a reasonable amount of time, and the formulation releases the active drug agent at the desired rate. The coating is generally performed by preparing a solution of the coating components in a solvent system, such as a mixture of acetone and water, and spraying the solution onto the cores in a solvent-ready, side-vented coating pan, in the case of tablets, or solvent-capable fluid bed, in the case of beads. In addition to formulation (compositional factors such as CA/PEG ratio and acetone/water ratio), the permeability of the membrane coating is also affected by parameters of the coating process, such as spray rate, and the nozzle-to-bed distance.

A typical composition for a high-permeability coating is CA 398-10 (6 wt %), PEG 3350 (4 wt %), water (23 wt %), and acetone (67 wt %); a medium-permeability coating is CA 398-10 (8 wt %), PEG 3350 (2 wt %), water (23 wt %), and acetone (67 wt %); and a low-permeability coating is CA 398-10 (9 wt %), PEG 3350 (1 wt %), water (23 wt %), and acetone (67 wt %). The final coating weight (without solvents) is typically from about 10 wt % to about 20 wt % of the core weight.

In a second embodiment, the invention provides controlled-release pharmaceutical formulations comprising a phosphodiesterase type 4D (PDE4D) inhibitor, or a pharmaceutically acceptable salt thereof, which formulations exhibit at least one of the following characteristics:
(i) a Tₘₐₓ of greater than about 1.5 hours;
(ii) less than about 80% of the PDE4D inhibitor, or the pharmaceutically acceptable salt thereof, is released in vivo at about 1.5 hours;
(iii) less than about 80% of the PDE4D inhibitor, or the pharmaceutically acceptable salt thereof, is released in vitro at about 1.5 hours; or
(iv) an in vivo delivery lag time prior to initiation of release of the PDE4D inhibitor, or the pharmaceutically acceptable salt thereof, of between about 0.5 hours and about four hours, wherein the formulations comprise a membrane-coated osmotic tablet comprising:
(a) a PDE4D inhibitor, or a pharmaceutically acceptable salt thereof;
(b) a hydroxyethyl cellulose having a weight average molecular weight of from about 300,000 to about 2,000,000;
(c) an osmagent; and
(d) a water-permeable membrane coating the osmotic tablet, wherein the membrane coating comprises at least one delivery port therethrough.

### ECS Formulations

The controlled-release formulations of the second embodiment of the invention utilize so-called “ECS” (extruding core system) technology, and may be prepared as disclosed in, for example, commonly-assigned U.S. Ser. No. 10/352,283, filed Jan. 27, 2003, the teachings of which are incorporated herein by reference in their entirety.

Controlled-release ECS formulations typically comprise a membrane-coated osmotic tablet comprising: (a) an active drug substance; (b) a hydroxyethyl cellulose; (c) an osmagent; and (d) a water-permeable membrane coating the osmotic tablet, wherein the membrane coating comprises at least one delivery port therethrough.

A hydroxyethylcellulose (HEC) is used as an entrainer, i.e., a carrier for the active drug agent as it is released from the core. The rheological properties of HEC also permit a more complete release of the active drug agent from the core into the surrounding fluid with minimal entrapment of the agent in the core at the end of delivery. Preferred HEC polymers useful in the present invention have a weight-average, molecular weight from about 300,000 to about 2,000,000 and a degree of polymerization from about 1,500 to about 6,700, preferably from 700,000 to 1,500,000 (degree of polymerization 3,500 to 5,000). The HEC polymer, when used at a molecular weight of 700,000 to 1,500,000, is typically present in the core in an amount from about 2.0% to about 20% by weight, preferably from about 3% to about 15%, more preferably from about 5% to about 10%. When the HEC is used in the 300,000 to 700,000 molecular weight range, the polymer is preferably present between 9 and 20%. The HEC can also be in a form designed to retard gel formation and thereby allow more uniform dissolution. Although several polymers have been disclosed in the art for use in osmotic tablets, only a small subset of those polymers provides a commercially useful means for drug delivery in a single-layer osmotic system suitable for limited-solubility drugs. Water-soluble polymers are added to keep drug particles suspended inside the dosage form before they exit through the delivery port(s). High viscosity polymers (i.e., having molecular weights up to about 2,000,000) are useful in preventing settling. However, the polymer in combination with the drug is extruded through the delivery port(s) under relatively low pressures. At a given extrusion pressure, the extrusion rate typically slows with increased viscosity. High molecular weight HEC in combination with the drug particles form high viscosity solutions with water but are still capable of being extruded from the tablets with a relatively low force. In contrast, other polymers and HECs having a low weight-average, molecular weight (i.e., less than about 300,000) do not form sufficiently viscous solutions inside the tablet core to allow complete delivery due to drug settling. An example of an HEC capable of forming solutions having a high viscosity yet still extrudable at low pressures is Natrosole 250H (high molecular weight HEC; Hercules Inc., Aqualon Division, Wilmington, DE; MW equal to about 1M and a degree of polymerization equal to about 3,700). Natrosole 250H provides effective drug delivery at concentrations as low as about 3% by weight of the core when combined with an osmagent. Natrosole 250H NF is a high-viscosity grade nonionic cellulose ether which is soluble in hot or cold water. The viscosity of a 1% solution of Natrosol® 250H using a Brookfield LVT (30 rpm) at 25° C. is between about 1,500 and about 2,500 cps.

The osmagent employed in the instant controlled-release ECS formulations may comprise those described hereinabove in the AMT controlled-release formulations.

Since the osmagent is typically the bulk excipient, the tableting properties of the osmagent are also considered. Typical tableting properties include flow (generally for direct compressed tablets) and mechanical properties. In the practice of the present invention, it has been determined that the optimum choice of osmagent can be accomplished by matching the ductility, tensile strength, and brittle fracture index (BFI) (Hiestand, et. al., Powder Technology, 38, 145 (1984)) of potential osmagents with the material properties of the drug. For some active drug substances, the binding of the substance to itself is sufficiently high that the osmagent serves to prevent the drug crystals from forming hard granules (during granulation), in which case, the use of fine grain osmagents is preferred. When the drug mechanical properties are combined with those of the osmagent and any other excipients, the resulting total blend properties determine the ability to form tablets with the blend. If the particle sizes of the drug, the osmagent(s), and other excipients are comparable (within about 25%) the blend properties will be a weighted average of the components. For a first approximation, the properties of the average should preferentially fall within the following ranges to achieve good tablets (i.e., tablets with low friability): ductility from about 100 to about 200 MPa; tensile strength from about 0.8 to about 2.0 MPa; and brittle fracture index (BFI) less than about 0.2. As mentioned above, these properties refer to a blend of the drug substance, the osmagent(s), and other excipients wherein the particle sizes for each of these components are comparable. In some cases, a binder may be desired to improve the binding properties of the tablet. Suitable binders include hydroxypropylcellulose (HPC) such as Klucele EXF (Hercules Inc., Aqualon Division; Wilmington, Del.) and Pharmacoat® 603 (Shin-Etsu Chemical Co., Japan).

Osmagents of different dissolution rates can sometimes be employed to influence how rapidly drug is initially delivered from the dosage form. For example, amorphous sugars such as Mannogeme EZ (SPI Pharma; Lewes, Del.) can provide faster delivery during the first couple of hours the dosage form is subjected to an aqueous environment. In some cases, the osmagent can serve as a bioavailability-enhancing additive. For example, some acids can solubilize some drugs in the GI tract as well as provide sufficient osmotic pressure for operation of the device. When this is possible, use of an osmagent as a solubilizer (bioavailability-enhancing additive) may be preferred since this allows for a maximum dose of active for a given tablet size. Preferred osmagents include salts, acids and sugars. Preferred salts include sodium chloride and potassium chloride. Preferred acids include ascorbic acid, benzoic acid, fumaric acid, citric acid, maleic acid, sebacic acid, sorbic acid, edipic acid, edetic acid, glutamic acid, p-tolunesulfonic acid and tartaric acid. A particularly preferred acid is tartaric acid. Preferred sugars include mannitol, sucrose, sorbitol, xylitol, lactose, dextrose and trehalose. A particularly preferred sugar is sorbitol. These osmagents can be used alone or as a combination of two or more osmagents. Sugars are preferred herein as osmagents. A particularly preferred osmagent is sorbitol. Sorbitol can be used as direct compress excipient (as with Neosorb® 30/60 DC; Roquette America, Inc.; Keokuk, Iowa) or in a smaller particle size suitable for use with granulations (such as Neosorb® P110).

Optional bioavailability-enhancing additives include additives known in the art to increase bioavailability of the active drug agent, such as solubilizing agents, additives that increase drug permeability in the GI tract, enzyme inhibitors, and the like. Suitable solubilizing additives include cyclodextrins and surfactants. Other additives that function to increase solubility include acidic or basic additives that solubilize a drug by altering the local pH in the GI tract to a pH where the drug solubility is greater than in the native system. Preferred additives are acids that both improve drug solubility in vivo and increase the osmotic pressure within the dosage form, thereby reducing or eliminating the need for additional osmagents. Preferred acids include ascorbic acid, benzoic acid, fumaric acid, citric acid, edetic acid, malic acid, sebacic acid, sorbic acid, adipic acid, glutamic acid, p-toluenesulfonic acid, and tartaric acid. Bioavailability-enhancing additives also include materials that inhibit enzymes that either degrade active agent or slow absorption by, for example, effecting an efflux mechanism. Another group of bioavailability-enhancing additives include materials that enable drug supersaturation in the GI tract. Such additives include enteric polymers as disclosed in PCT International Application Publication No. WO 01/47495 Al, EP 1 027 886 A2, and EP 1 027 885 A2. Particularly preferred polymers of this type include HMPCAS and CAP.

Acids or bases can also function to mediate the pH within the core during use and thereby reduce the drug delivery sensitivity to the pH of the use environment. In particular, it has been observed that for some drugs, their dispersability depends on the pH of the dispersing water. For the dosage form of the present invention to function effectively, the drug must disperse, and thereby be entrained in the exiting fluid. For drugs that have pH sensitivity in their dispersability, it has been determined that the addition of about 5% and about 25% by weight of a soluble acid or base (depending on the pH for optimal dispersability of the drug) allows for drug delivery to be essentially independent of the external environmental pH. A particularly preferred acid useful for basic drugs is tartaric acid. Preferred bases useful for acidic drugs include alkaline metal and alkaline earth salts of carbonate, bicarbonate and oxide, sodium phosphate (di-basic and mono-basic), triazine base, guanidine, and N-methyl glucamine.

The ECS controlled-release formulations may optionally comprise disintegrants, such as sodium starch glycolate (e.g, Explotab® CLV), microcrystalline cellulose (e.g., Avicel®), microcrystalline silicified cellulose (e.g., ProSolv®), or croscarmellose sodium (e.g., Ac-Di-Sol®), and similar disintegrants known in the art. Additionally, non-gelling, non-swelling disintegrants, such as resins may be employed. A generally preferred resin is Amberlite® IRP 88 (Rohm & Haas; Philadelphia, Pa.). When employed, the distintegrant is present in amounts ranging from about 1% and about 25% w/w of the core composition, preferably from about 1% and about 15%. The instant ECS formulations may further optionally comprise dispersing aids, such as low weight-average molecular weight polar polymers, such as carbomers or polyvinyl alcohols, surfactants, such as sodium dodecylsulfate, or agents designed to make the pH inside the tablet core independent of the dissolution medium, such as tartaric acid. If employed, the acid preferably present at between about 1% and about 50% w/w of the core components, preferably between about 1% and about 30%. Another preferred dispersing aid is a poloxamer, i.e., a block copolymer of polyethylene oxide (PEO) and polypropylene oxide as disclosed in “Handbook Of Pharmaceutical Excipients”, 3^{rd} Edition, (American Pharmaceutical Association) 2000, pp.386-388. The poloxamer is most effective when in intimate contact with the drug. Such intimate contact can be achieved by, for example, coating a solution of the poloxamer onto drug crystals. Poloxamer, when used, is preferably present at a level between 1-20% by weight of the core, preferably between 1-10% by weight of the core. A generally preferred poloxamer is Pluronice F127 (BASF Corp.; Mt. Olive, N.J.). Finally, the ECS tablet core may further comprise one or more pharmaceutically acceptable excipients, carriers, or diluents. Excipients are typically selected to provide good compression profiles under direct compression. For example, a lubricant is typically used to prevent the tablet and punches from sticking in the die. Suitable lubricants comprise talc, magnesium or calcium stearate, stearic acid, light anyhdrous silicic acid, or hydrogenated vegetable oils. A generally preferred lubricant is magnesium stearate. Other useful additives include materials such as surface active agents (e.g., cetyl alcohol, glycerol monostearate, and sodium lauryl sulfate); adsorptive carriers, such as kaolin and bentonite; preservatives; sweeteners; coloring agents; flavoring agents, such as citric acid, menthol, glycine, or orange powder; stabilizers, such as citric acid, sodium citrate, and acetic acid. Typically, such additives are present at levels below about 10% of the core weight.

The ECS core may be prepared according to known methods. For example, the core components are generally mixed together, compressed into a solid form, the core is overcoated with a water-permeable core, and then a delivery means through the water-permeable core is provided (e.g., a hole is drilled into the coating to form an orifice. In some instances, the components are simply mixed together and then compressed directly. However, it may be desirable for some formulations to be granulated by conventional techniques, followed by subsequent compression into a solid form. The tablet core may be prepared by standard tableting processes, such as by using a conventional rotary tablet press.

After compression, the tablet cores are ejected from the die. The cores are then overcoated with a water-permeable coating using standard procedures well-known to those skilled in the art. The water-permeable coating contains at least one delivery port (passageway) through which the drug is substantially delivered from the device. Preferably, the drug is delivered through the passageway as opposed to delivery primarily via permeation through the coating material itself. The term “delivery port” refers to an opening or pore whether made mechanically, by laser drilling, in situ during use or by rupture during use. The delivery port can extend into the core. However, since drilling a significant distance into the core can lead to loss of potency (and potential degradation if laser drilled), it is preferred that the penetration depth be less than 10% of the diameter of the tablet at that point, preferably less than 5%. Preferably, the delivery port is provided by laser or mechanical drilling. The water-permeable coating can be applied by any conventional film coating process well known to those skilled in the art, for example, by spray coating in a pan or fluidized bed coating. The water-permeable coating is generally present in an amount ranging from about 3 wt % to about 30 wt %, preferably from about 6 wt % to about 15 wt %, relative to the core weight.

A preferred form of the coating is a water-permeable polymeric membrane. The delivery port (s) may be formed either prior to or during use. The thickness of the polymeric membrane generally varies between about 20 μm and about 800 μm, and is preferably in the range of about 100 μm to about 500 μm. The size of the delivery port will be determined by the particle size of the drug, the number of delivery ports in the device, and the desired delivery rate of the drug during operation. A typical delivery port has a diameter from about 25 μm to about 2000 μm, preferably from about 300 μm to about 1200 μm, more preferably from about 400 μm to about 1000 μm. The delivery port(s) may be formed post-coating by mechanical or laser drilling, or may be formed in situ by rupture of the coatings. Rupture of the coating may be controlled by intentionally incorporating a relatively small weak portion into the coating. Delivery ports may also be formed in situ by erosion of a plug of water-soluble material or by rupture of a thinner portion of the coating over an indentation in the core. Multiple holes can be made in the coating, however, oblong-shaped tablets having a single hole at one end of the tablet are generally preferred.

Specific examples of suitable polymers (or crosslinked versions) useful in forming the coating include plasticized, unplasticized and reinforced CA, cellulose diacetate, cellulose triacetate, CA propionate, cellulose nitrate, cellulose acetate butyrate (CAB), CA ethyl carbamate, CAP, CA methyl carbamate, CA succinate, CAT, CA dimethylaminoacetate, CA ethyl carbonate, CA chloroacetate, CA ethyl oxalate, CA methyl sulfonate, CA butyl sulfonate, CA p-toluenesulfonate, agar acetate, amylose triacetate, beta-glucan acetate, beta-glucan triacetate, acetaldehyde dimethyl acetate, triacetate of locust bean gum, hydroxlated ethylene-vinylacetate, ethyl cellulose (EC), PEG, PPG, PEG/PPG copolymers, polyvinylpyrrolidone (PVP), HEC, hydroxypropyl cellulose (HPC), carboxymethyl cellulose (CMC), carboxymethylethyl cellulose (CMEC), HPMC, hydroxypropylmethyl cellulose propionate (HPMCP), HPMCAS, poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes.

A preferred coating composition comprises a cellulosic polymer, in particular cellulose ethers, cellulose esters and cellulose ester-ethers, i.e., cellulosic derivatives having a mixture of ester and ether substituents, such as HPMCP. Another preferred class of coating materials comprises poly(acrylic) acids and esters, poly(methacrylic) acids and esters, and copolymers thereof.

A more preferred coating composition comprises CA. Preferred cellulose acetates are those with acetyl contents between 35% and 45% and number-average, molecular weights (MWn) between 30,000 and 70,000. An even more preferred coating comprises a cellulosic polymer and PEG. A most preferred coating comprises cellulose acetate and PEG. A preferred PEG has a weight-average molecular weight from about 2,000 to about 5,000, more preferably between 3,000 and 4,000.

The coating process is conducted in conventional fashion, typically by dissolving the coating material in a solvent and then coating by dipping, fluid bed coating, spray-coating or preferably by pan-coating. A preferred coating solution contains about 5% to about 15% w/w polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. The use of water based latex or pseudo-latex dispersions are also possible for the coating. Such coatings are preferred due to the manufacturing advantages of avoiding organic solvents and potential environmental challenges therein. Pore-formers and non-solvents (such as water, glycerol and ethanol) or plasticizers (such as diethyl phthalate and triacetin) may also be added in any amount as long as the polymer remains soluble at the spray temperature. Pore-formers and their use in fabricating coatings are described in U.S. Pat. No. 5,612,059, the teachings of which are incorporated herein by reference. In general, more water-soluble additives (such as PEG) increase the water-permeability of the coating (and thereby the drug delivery rate) while water insoluble additives (such as triacetin) decrease the rate of drug delivery.

The position and number of delivery ports can have a significant impact on the drug delivery rate and residual amount of drug remaining after 24 hours in a dissolution medium. In particular, a single delivery port drilled on the band of the tablet generally provides superior performance. For oblong or caplet-shaped tablets, the delivery port is preferably made on the band at one tip of the tablet (i.e., coincident with the major axis). The advantage of a delivery port on the end for oblong or caplet-shaped tablets is believed to be due to the ability of the shape to focus the final percentage of extrudable material to the exit hole.

It may be desirable to provide an additional coating or coatings on the inside or outside of the water-permeable coating. Coatings underneath the water-permeable coating are preferably permeable to water. Such coatings can serve to improve adhesion of the water-permeable coating to the tablet core, or to provide a chemical and/or act as a physical barrier between the core and the water-permeable coating. A barrier coating can insulate the core during coating to the water-permeable coating from, for example, the coating solvent or from migration of a plasticizer (e.g., PEG) during storage. External coatings can be cosmetic to help with product identification and marketing, and improve mouth feel and swallowability. Such coatings can also be functional. Examples of such functional coatings include enteric coatings (i.e., coatings designed to dissolve in certain regions in the gastrointestinal tract) and opacifying coatings (designed to block light from reaching a light-sensitive drug). Other product identifying features can also be added to the top of the coating. Examples include, but are not limited to, printing and embossing of identifying information. The additional coating can also contain an active pharmaceutical ingredient, either the same or different from that in the core. This can provide for combination drug delivery and/or allow for specific pharmacokinetics (e.g., pulsatile). Such a coating can be film coated with an appropriate binder onto the tablet core. In addition, active material can be compression coated onto the tablet surface. In many cases, this compression coating can be facilitated by use of a compressible film coat.

In a third embodiment, the invention provides controlled-release pharmaceutical formulations comprising a phosphodiesterase type 4D (PDE4D) inhibitor, or a pharmaceutically acceptable salt thereof, which formulations exhibit at least one of the following characteristics:
(i) a Tₘₐₓ of greater than about 1.5 hours;
(ii) less than about 80% of the PDE4D inhibitor, or the pharmaceutically acceptable salt thereof, is released in vivo at about 1.5 hours;
(iii) less than about 80% of the PDE4D inhibitor, or the pharmaceutically acceptable salt thereof, is released in vitro at about 1.5 hours; or
(iv) an in vivo delivery lag time prior to initiation of release of the PDE4D inhibitor, or the pharmaceutically acceptable salt thereof, of between about 0.5 hours and about four hours, wherein the formulations comprise:
(a) a PDE4D inhibitor, or a pharmaceutically acceptable salt thereof; and
(b) a polymer that moderates release of the PDE4D inhibitor, or the pharmaceutically acceptable salt thereof.

### Matrix Tablet Formulations

The controlled-release formulations of the third embodiment of the invention utilize so-called “matrix tablet” technology and will be well-known to one of ordinary skill in the relevant art. See, for example, K. Takada, et al., “Oral Drug Delivery, Traditional”, Encyclopedia of Controlled Drug Delivery, Vol. 2, Edith Mathiowitz, ed., Wiley, (1999). Although a diversity of matrix tablet formulations are known in the relevant art, generally preferred matrix tablet formulations useful in the practice of the present invention, comprise hydrophilic, hydrophobic, or plastic matrix tablet formulations.

Typical hydrophilic matrix tablet formulations comprise an active drug substance, and a hydrophilic polymer that swells in the presence of water, thereby forming a gel through which the active drug substance diffuses. The active drug agent is also released by an erosion mechanism. Exemplary hydrophilic polymers comprise HPMC, PEO, polyacrylic acid, polyvinyl alcohol (PVA), HPC, MC, carboxymethyl cellulose sodium, PVP, poly(2-hydroxyethyl methacrylate), and the like. Crosslinked acrylic acid-based hydrophilic polymers and copolymers such as Carbopol® (Noveon Inc.; Cleveland, Ohio) can also be used in matrix tablet formulations. Optionally, hydrophilic matrix tablet formulations may further comprise tableting aids, such as binders, fillers, and the like. Exemplary tableting aids include sugars, such as such as lactose and xylitol; dibasic calcium phosphate; and polymers, such as microcrystalline cellulose, HPC, MC, and HPMC. The hydrophilic matrix tablet formulations may further comprise lubricants, such as magnesium stearate, as well as the concentration-enhancing polymers and/or solubilizers described hereinabove.

Typical hydrophobic matrix tablet formulations comprise an active drug substance and a wax, such as carnauba wax, or other low-melting hydrophobic material, such as glyceryl behenate. Other hydrophobic matrix materials include fatty alcohols, fatty acids, and fatty esters. EC can also be used as an inert, hydrophobic polymer in matrix tablets. Optionally, hydrophobic matrix tablet formulations may further comprise tableting aids, such as binders, fillers, and lubricants, including those described hereinabove for hydrophilic matrix tablet formulations, as well as the concentration-enhancing polymers and/or solubilizers disclosed hereinabove.

Typical plastic matrix tablet formulations comprise an active drug substance and an inert pharmaceutical polymer, such as polyvinyl chloride (PVC), polyvinyl acetate, and methyl methacylate. Optionally, plastic matrix tablet formulations may further comprise tableting aids, such as binders, fillers, and lubricants, including those described hereinabove for hydrophilic matrix tablet formulations, as well as the concentration-enhancing polymers and/or solubilizers disclosed hereinabove.

Hydrophilic, hydrophobic, and plastic matrix tablet formulations may be manufactured by methods well-known in the relevant art, including direct compression, dry or wet granulation followed by compression, melt-granulation, followed by compression, and the like. The tablets may optionally be coated, for example, color-coated for appearance, product differentiation, taste-masking, and the like.

Selection of polymers an their levels in the formulation to moderate the release of the active drug agent from a matrix tablet formulation, and methods of manufacturing matrix tablet formulations, are well-known in the art. In general, the matrix material, such as HPMC, can comprise from about 15% to about 55% w/w of the formulation.

In a fourth embodiment, the invention provides controlled-release pharmaceutical formulations comprising a phosphodiesterase type 4D (PDE4D) inhibitor, or a pharmaceutically acceptable salt thereof, which formulations exhibit at least one of the following characteristics:
(i) a Tₘₐₓ of greater than about 1.5 hours;
(ii) less than about 80% of the PDE4D inhibitor, or the pharmaceutically acceptable salt thereof, is released in vivo at about 1.5 hours;
(iii) less than about 80% of the PDE4D inhibitor, or the pharmaceutically acceptable salt thereof, is released in vitro at about 1.5 hours; or
(iv) an in vivo delivery lag time prior to initiation of release of the PDE4D inhibitor, or the pharmaceutically acceptable salt thereof, of between about 0.5 hours and about four hours, wherein the formulations comprise:
(a) a PDE4D inhibitor, or a pharmaceutically acceptable salt thereof;
(b) a carrier; and
(c) a dissolution-enhancing agent.

### Multiparticulate Formulations

The multiparticulate controlled-release formulations of the fourth embodiment are well-known dosage forms that comprise a multiplicity of particles whose totality represents the intended therapeutically useful dose of a drug. When taken orally, multiparticulates generally disperse freely in the gastrointestinal tract, exit relatively rapidly and reproducibly from the stomach, maximize absorption, and minimize side effects. See, for example, Multiparticulate Oral Drug Delivery (Marcel Dekker, 1994), and Pharmaceutical Pelletization Technology (Marcel Dekker, 1989). Typical multiparticulate formulations comprise an active drug substance, a dissolution-enhancing agent, and a carrier.

Dissolution-enhancing agents typically comprise from about 0.1% to about 30% w/w, preferably from about 1% to about 15% w/w of the formulation, based on the total weight of the multiparticulate. Examples of dissolution-enhancing agents may comprise dispersing or emulsifying agents, such as poloxamers (polyoxyethylene or polyoxypropylene co-polymers), such as the PLURONIC® and LUTROL® (BASF Corp., Mt. Olive, N.J.) series; ether-substituted cellulosics, such as HPC and HPMC; polyoxyethylene alkyl esters and ethers, such as BRIJ® and CHREMOPHOR® A; polyoxyethylene castor oil derivatives, such as CHREMOPHOR® RH40; polyoxyethylene sorbitan fatty acid esters, such as TWEEN® 80 and CAPMUL® POE-O; sorbitan esters, such as CAPMUL-O® and SPAN® 80; alkyl sulfates, such as sodium lauryl sulfate; sugars, such as glucose, sucrose, xylitol, sorbitol, and maltitol; alcohols, such as stearyl alcohol or cetyl alcohol, and low molecular weight (e.g., less than about 10,000 daltons) polyethylene glycol; salts, such as sodium chloride, potassium chloride, lithium chloride, calcium chloride, magnesium chloride, sodium sulfate, potassium sulfate, sodium carbonate, magnesium sulfate, and potassium phosphate; and amino acids, such as alanine and glycine. A generally preferred class of dissolution enhancers comprises poloxamers.

The carrier, which may comprise a blend of species, will generally comprise about 20% to about 90% w/w of the multiparticulate, based on the total mass of the multiparticulate. The carrier, in conjunction with the dissolution enhancer, functions as a matrix for the mutliparticulate, or to control the rate of release of the drug therefrom. Preferably, the carrier comprises a substance different from the dissolution enhancer.

Generally, carriers are classified into four categories: (1) non-reactive, (2) low reactivity, (3) moderate reactivity, and (4) highly reactive.

Non-reactive carriers generally have no acid or ester substituents and are free from impurities that contain acids or esters. Generally, non-reactive materials will have an acid/ester concentration of less than 0.0001 meq/g of carrier. Non-reactive carriers are normally rare and must be highly purified. In addition, non-reactive carriers are often hydrocarbons, since the presence of other materials in the carrier can lead to acid or ester impurities. Examples of non-reactive carriers include highly-purified forms of hydrocarbons such as synthetic wax, microcrystalline wax, and paraffin wax.

Low reactivity carriers also do not have acid or ester substituents, but often contain small amounts of impurities or degradation products that contain acid or ester substituents. Generally, low reactive carriers have an acid/ester concentration of less than about 0.1 meq/g of carrier. Examples of low reactivity carriers comprise long-chain alcohols, such as stearyl alcohol, cetyl alcohol, and PEG; dispersing or emulsifying agents, such as poloxamers; ethers, such as PEO and polyoxyethylene alkyl ethers; ether-substituted cellulosics, such as microcrystalline cellulose, HPC, HPMC, and EC; sugars, such as glucose, xylitol, sorbitol, and maltitol; and salts, such as sodium chloride, potassium chloride, lithium chloride, calcium chloride, magnesium chloride, sodium sulfate, potassium sulfate, sodium carbonate, magnesium sulfate, and potassium phosphate.

Moderate reactivity carriers often contain acid or ester substituents, but relatively few as compared to the molecular weight of the carrier. Generally, the moderate reactivity carriers have an acid/ester concentration of about 0.1 to about 3.5 meq/g of carrier. Examples include long-chain fatty acid esters, such as glyceryl mono-oleate, glyceryl mono-stearate, glyceryl palmitostearate, polyethoxylated castor oil derivatives, glyceryl di-behenate, and mixtures of mono-, di-, and tri-alkyl glycerides, including mixtures of glyceryl mono-, di-, and tri-behenate, glyceryl tri-stearate, glyceryl tri-palmitate, and hydrogenated vegetable oils; glycolized fatty acid esters, such as PEG stearate and PEG di-stearate; and waxes, such as carnauba wax and white and yellow beeswax.

Highly reactive carriers usually have several acid or ester substituents or low molecular weights. Generally, highly reactive carriers have an acid/ester concentration of more than about 3.5 meq/g of carrier. Examples include carboxylic acids, such as stearic acid, benzoic acid, citric acid, fumaric acid, lactic acid, and maleic acid; short-to-medium chain fatty acid esters, such as isopropyl palmitate, isopropyl myristate, triethyl citrate, lecithin, and di-butyl sebacate; ester-substituted cellulosics, such as CA, CAP, MPMCP, CAT, and HPMCAS; and acid or ester functionalized polymethacrylates and polyacrylates. Generally, highly reactive carriers are preferably only used in combination with a carrier with lower reactivity such that the total amount of acid and/or ester groups on the carrier used in the particulate is low.

If desired and/or appropriate, the multiparticulate formulations may further comprise the concentration-enhancing polymers, solubilizers, and/or conventional excipients disclosed hereinabove.

Preferred processes to form controlled release multiparticulates include thermal-based processes such as melt- and spray-congealing; liquid-based processes, such as extrusion spheronization, wet granulation, spray-coating and spray-drying and other granulation processes, such as dry granulation and melt granulation.

An especially preferred process to form controlled release multiparticulates includes they melt-spray-congeal process comprising the steps (a) forming a molten mixture comprising the active drug substance, at least one pharmaceutically acceptable carrier, and at least one dissolution enhancer, (b) delivering the molten mixture of step (a) to an atomizing means to form droplets from the molten mixture, and (c) congealing the droplets from step (b) to form multiparticulates.

Virtually any process can be used to form the molten mixture. An especially preferred method comprises the use of an extruder, such as a single-screw or twin-screw extruder, which produces a molten mixture that can be directed to the atomizer. The atomization is accomplished in one of several ways, including: (1) by “pressure” or single-fluid nozzles, (2) by two-fluid nozzles, (3) by centrifugal or spinning-disk atomizers, (4) by ultrasonic nozzles, or (5) by mechanical vibrating nozzles. Detailed descriptions of atomization processes can be found in Lefebvre, Atomization and Sprays (1989) or in Perry's Chemical Engineers' Handbook, 7^{th} Ed., (1997). Once the molten mixture has been atomized, the droplets are congealed, typically by contact with a gas or liquid at a temperature below the solidification temperature of the droplets.

Although any PDE4D inhibitor, or pharmaceutically acceptable salt thereof, may be employed in the controlled-release formulations and methods of the present invention, preferred PDE4D inhibitors comprise the compounds (R)-2-[4-({[2-(benzo[1,3]dioxol-5-yloxy)-pyridine-3-carbonyl]-amino}-methyl)-3-fluoro-phenoxy]-propionic acid, and 2-(4-fluorophenoxy)-N-[4-(1-hydroxy-1-methyl-ethyl)-benzyl]-nicotinamide, i.e., the compounds of structural formulae (I) and (la) respectively hereinbelow, and the pharmaceutically acceptable salts thereof.

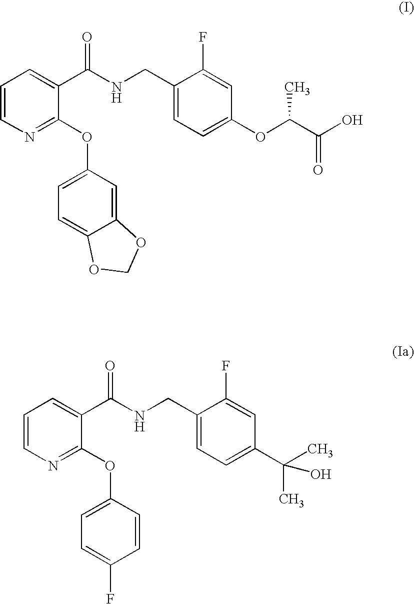

The preferred PDE4D inhibitor (I), and the pharmaceutically acceptable salts thereof, may be conveniently prepared as disclosed in PCT International Application Publication No. WO 2002/060896.

A preferred method for preparing compound (I) is illustrated hereinbelow in Scheme 1. Preferred methods for preparing the nicotinic acid (III) and amine intermediates (IV) are depicted hereinbelow in Schemes 2 and 3 respectively.

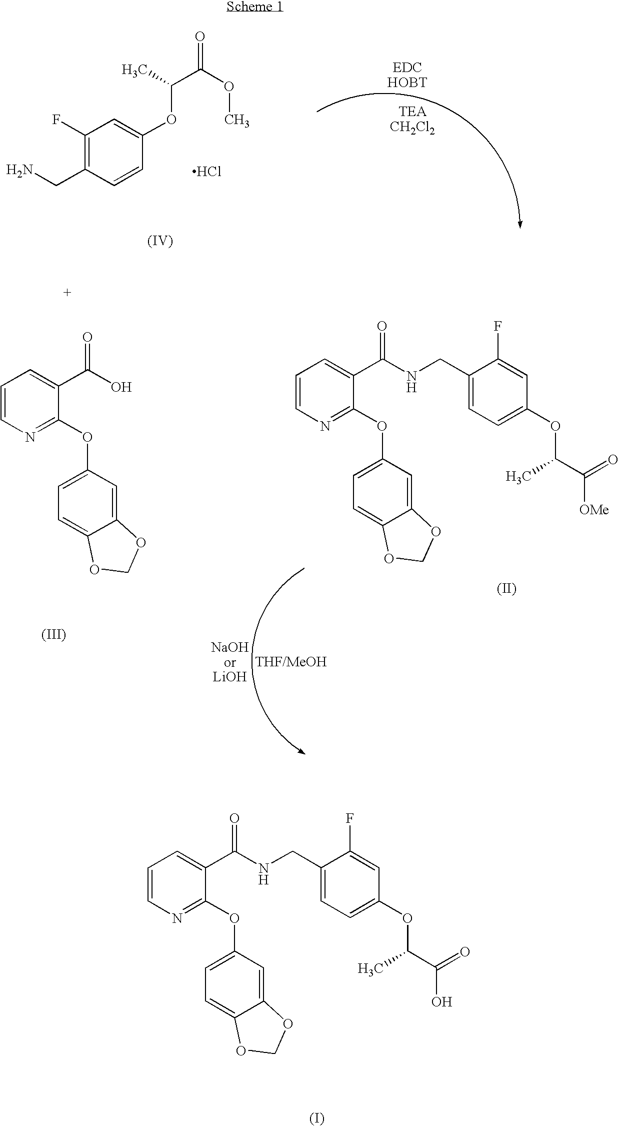

In Scheme I hereinabove, 2-(benzo-[1,3]dioxolo-5-yloxy)-nicotinic acid (III) is condensed with (R)-2-(4-aminomethyl-3-fluoro-phenoxy)-propionic acid methyl ester hydrochloride (IV), in the presence of a coupling agent, such as 1,1′-carbonyldiimidazole (CDI), 1-(3-dimethylaminopropyl)-3-ethylcarbodiimide (EDC), or 1,3-dicyclohexylcarbodiimide (DCC), and an organic base, such as triethylamine. Such condensation is typically effected in an aprotic solvent, such as N,N-dimethylformamide (DMF), or dichloromethane, preferably at ambient temperature. The methyl ester (II) so formed is then saponified with aqueous base, for example, lithium hydroxide or sodium hydroxide, in a protic solvent, preferably methanol, or a mixture or tetrahydrofuran (THF)/methanol, to afford (I).

The pharmaceutically acceptable salts of (I), preferably the basic addition salts, may be prepared according to conventional methods. For example, the preferred basic addition salts may be prepared by contacting (I) with a stoichiometric amount of an appropriate organic or inorganic base to provide the corresponding basic addition salt. Inorganic basic addition salts of the present invention include, but are not limited to, aluminum, ammonium, calcium, copper, ferric, ferrous, lithium, magnesium, manganic, manganous, potassium, sodium, and zinc salts. Preferred among the recited inorganic basic addition salts are ammonium; the alkali metal salts sodium and potassium; and the alkaline earth metal salts calcium and magnesium. Salts of (I) derived from non-toxic organic bases include, but are not limited to, salts of primary, secondary, and tertiary amines, substituted amines including naturally-occurring substituted amines, cyclic amines, and basic ion exchange resins, e.g., arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

A preferred method for preparing intermediate (III) is illustrated hereinbelow in Scheme 2.

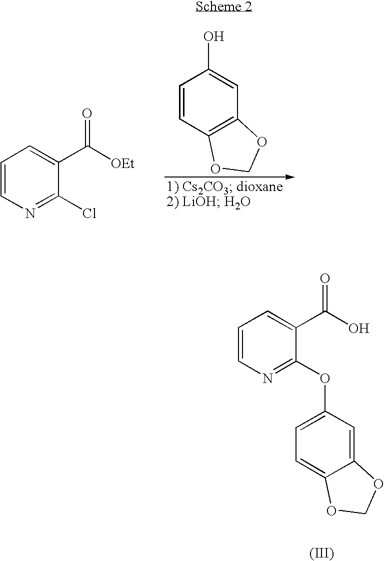

In Scheme 2, Step 1, hereinabove, 2-chloro-nicotinic acid ethyl ester is condensed with benzo[1,3]dioxol-5-ol (sesamol) in the presence of an inorganic base, such as potassium carbonate or cesium carbonate. Such condensation is typically effected in an aprotic solvent, such as DMF, THF, or dioxane at elevated temperature. Preferably, the condensation is effected in the presence of cesium carbonate in refluxing dioxane. In Scheme 2, Step 2, intermediate (III) is most conveniently prepared by in situ hydrolysis of the resulting ethyl ester precursor of (III) obtained from the condensation of 2-chloro-nicotinic acid ethyl ester and benzo[1,3]dioxol-5-ol. However, one of ordinary skill in the art will appreciate that such ethyl ester precursor of (III) may, if desired and/or appropriate, be isolated and hydrolyzed in a separate step.

A preferred method for preparing the amine intermediate (IV) is illustrated hereinbelow in Scheme 3.

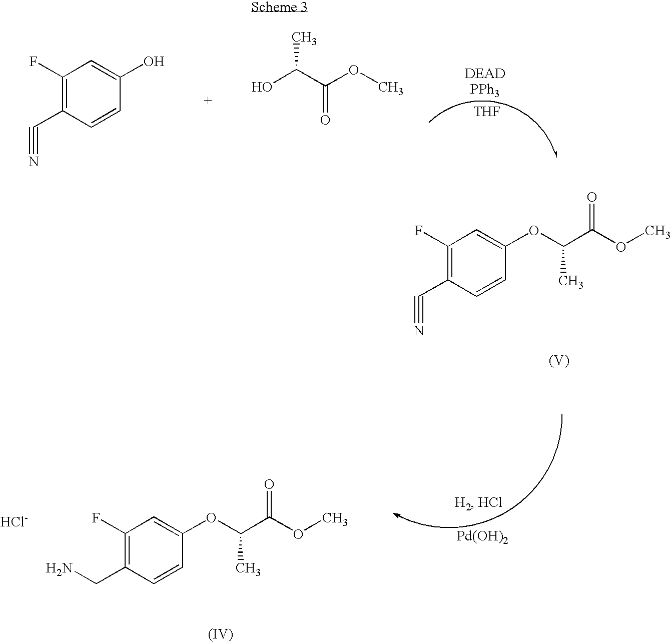

In Scheme 3 hereinabove, 2-fluoro4-hydroxybenzonitrile is condensed with methyl (S)-(−)-lactate via the so-called Mitsunobu reaction to afford benzonitrile (V). Such condensation is typically effected in the presence of a dehydrating reagent, for example, a stoichiometric amount of a diazocarboxyl compound, such as diethyl azodicarboxylate, and a phosphine, for example, triphenylphosphine. The reaction is effected in a reaction-inert, aprotic solvent, such as THF. The functionalized benzonitrile (V) so formed is then reduced, preferably by catalytic hydrogenation with palladium hydroxide in a protic solvent, such as methanol. The resulting free amine is most conveniently isolated as the acid addition salt thereof. A preferred acid addition salt comprises the hydrochloride salt (IV). The acid addition salts, including the preferred hydrochloride addition salt (IV), may be prepared according to known methods. The preferred hydrochloride addition salt (IV) is preferably prepared by performing the catalytic hydrogenation of benzonitrile (V) in the presence of at least one molar equivalent of hydrochloric acid.

The preferred PDE4D inhibitor of formula (Ia), and the pharmaceutically acceptable salts thereof, may be prepared as disclosed in commonly-assigned U.S. Pat. No. 6,380,218, the disclosure of which is incorporated herein by reference in its entirety.

A convenient method for preparing compound (la) is disclosed hereinbelow in Scheme 4.

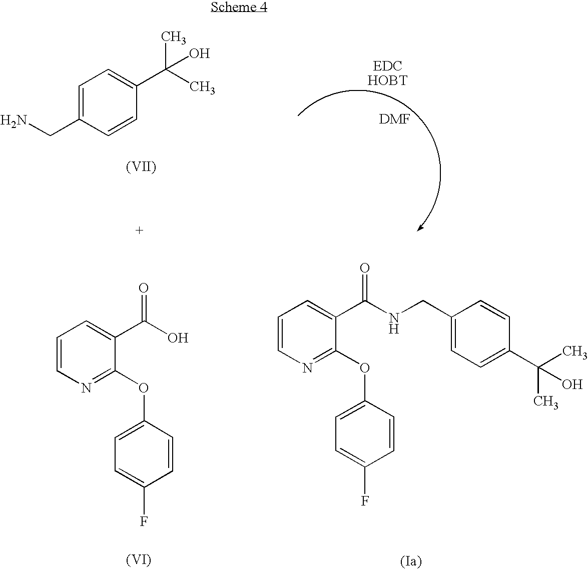

In Scheme 4 above, 2-(4-fluoro-phenoxy)-nicotinic acid (VI) and 2-(4-aminomethyl-phenyl)-propan-2-ol (VII) may be coupled according to the general methods described hereinabove in Scheme I. Preferably, the coupling reaction is effected at room temperature using EDC in dry DMF. Nicotinic acid intermediate (VI) is conveniently prepared by condensing 4-fluorophenol with 2-chloronicotinic acid in the presence of an inorganic base, preferably sodium hydride, in an aprotic solvent, preferably DMF, at reflux temperature. Amine intermediate (VII) is conveniently prepared by reducing 4-(1-hydroxy-1-methyl-ethyl)-benzonitrile with a hydride reducing agent, preferably lithium aluminum hydride, in an aprotic solvent, preferably THF. The 4-(1-hydroxy-1-methyl-ethyl)-benzonitrile starting material is prepared by reacting 4-cyanoacetophenone with methyl magnesium chloride in dry THF at −78° C., followed by conventional work-up.

The pharmaceutically acceptable salts of (la), preferably the acid addition salts, may also be prepared according to conventional methods. For example, the preferred acid addition salts may be prepared by contacting (Ia) with a stoichiometric amount of an appropriate inorganic or organic acid to provide the corresponding acid addition salt. Inorganic acid addition salts may comprise, for example, the hydrochloric, hydrobromic, nitric, sulfuric, and phosphate addition salts. Organic acid addition salts may comprise, for example, the acetate, besylate, citrate, fumarate, tartrate, and tosylate addition salts.

As employed herein, the term Tₘₐₓ is a well-known pharmacokinetic parameter obtained from the plasma concentration vs. time profiles.

The invention further provides methods of treating disorders and conditions mediated by the PDE4D isozyme, which comprise administering to a mammal in need of such treatment a therapeutically effective amount of a PDE4D inhibitor, or a pharmaceutically acceptable salt thereof, in a controlled-release formulation of the present invention. Preferably, the PDE4D inhibitor comprises the compound (R)-2-[4-({[2-(benzo[1,3]dioxol-5-yloxy)-pyridine-3-carbonyl]-amino[-methyl)-3-fluoro-phenoxy]-propionic acid, or a pharmaceutically acceptable salt thereof, or the compound 2-(4-fluorophenoxy)-N-[4-(1-hydroxy-1-methyl-ethyl)-benzyl]-nicotinamide, or a pharmaceutically acceptable salt thereof.

Preferred disorders and conditions treatable according to the present methods are selected from the group consisting of:
(a) inflammatory diseases and conditions selected from the group consisting of joint inflammation, rheumatoid arthritis, rheumatoid spondylitis, osteoarthritis, inflammatory bowel disease, ulcerative colitis, chronic glomerulonephritis, dermatitis, and Crohn's Disease;
(b) respiratory diseases and conditions selected from the group consisting of asthma, acute respiratory distress syndrome, chronic obstructive pulmonary disease, bronchitis, chronic obstructive airway disease, and silicosis;
(c) infectious diseases and conditions selected from the group consisting of sepsis, septic shock, endotoxic shock, gram negative sepsis, toxic shock syndrome, fever and myalgias due to bacterial, viral or fungal infection, and influenza;
(d) immune diseases and conditions selected from the group consisting of autoimmune diabetes, systemic lupus erythematosis, graft v. host reaction, allograft rejections, multiple sclerosis, psoriasis, and allergic rhinitis; and
(e) other diseases and conditions selected from the group consisting of bone resorption diseases, reperfusion injury, cachexia secondary to infection or malignancy, cachexia secondary to human immunodeficiency syndrome (AIDS), human immunodeficiency virus (HIV), infection, or AIDS related complex (ARC), keloid formation, scar tissue formation, Type 1 diabetes mellitus, and leukemia.

Especially preferred disorders and conditions treatable according to the present methods are asthma, acute respiratory distress syndrome, chronic obstructive pulmonary disease, bronchitis, chronic obstructive airway disease, and silicosis.

Typically, dosages of PDE4D inhibitors, or the pharmaceutically acceptable salts thereof, comprising the instant controlled-release formulations range from about 0.1 μg to about 50.0 mg/kg of body mass per day, preferably from about 5.0 μg to about 5.0 mg/kg of body mass per day, more preferably from about 10.0 μg to about 1.0 mg/kg of body mass per day, and, most preferably, from about 20.0 μg/kg to about 0.5 mg/kg of body mass per day. Some variability, however, some variability in the general dosage ranges may be required depending upon the age and weight of the patient being treated, the particular PDE4D inhibitor being administered, the nature and kind of concurrent therapy, if any, the frequency of treatment and the nature of the effect desired, and the like. The determination of dosage ranges and optimal dosages for a particular patient is well within the ability of one of ordinary skill in the art having the benefit of the instant disclosure.

The invention further provides methods of reducing PDE4D inhibitor treatment-induced nausea and/or emesis in a mammal which comprise administering the PDE4D inhibitor, or a pharmaceutically acceptable salt thereof, to the mammal in the form of a controlled-release formulation of the present invention. Preferred PDE4D inhibitors useful in such methods comprise the compounds of structural formulae (I) and (Ia) hereinbelow, and the pharmaceutically acceptable salts thereof.

### Experimental

### Chemical Syntheses

With reference to the synthetic outlines depicted in Schemes 1, 2, and 3 hereinabove, compound (I) is prepared utilizing the intermediates of the following Examples. Other synthetic variations will be known, or apparent in light of the instant disclosure, to one of ordinary skill in the art. Unless otherwise noted, all reactants were obtained commercially.

### Preparation 1

### 2-(Benzo-[1,3]dioxolo-5-yloxy)-nicotinic acid (III)

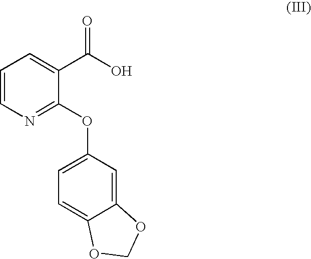

2-Chloro-nicotinic acid ethyl ester (10 g), benzo[1,3]dioxol-5-ol (sesamol, 8.2 g), and cesium carbonate (21 g) were mixed in anhydrous dioxane (40 mL) and the resulting slurry was heated to reflux for 16 hr. In a separate flask, lithium hydroxide (12.9 g) was dissolved in water (80 mL) with warming and then added to the refluxing mixture, which was heated for an additional four hr. The mixture was cooled to ambient temperature and then concentrated in vacuo to remove the dioxane. Concentrated hydrochloric acid was added dropwise until the pH=3. The acidified solution was then extracted with ethyl acetate (7×100 mL) to yield the crude product, which was recrystallized from ethyl acetate to yield the purified title compound (10. 8 g).

¹H NMR (CD₃OD): δ 8.28 (dd, J=8 and 2 Hz, 2H), 7.13 (m, 1H), 6.79 (d, J=8 Hz, 1H), 6.62 (s, J=2 Hz, 1H), 6.53 (dd, J=8 and 2 Hz, 1H), 5.95 (s, 2H).

### Preparation 2

### (R)-2-(4-Cyano-3-fluoro-phenoxy)-propionic acid methyl ester (V)

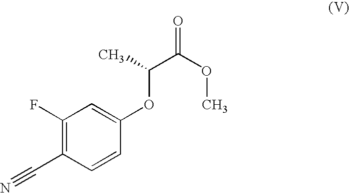

To a stirred solution of 2-fluoro-4-hydroxybenzonitrile (0.2 g, 1.5 mmol), methyl (S)-(−)-lactate (0.14 mL, 1.5 mmol) and triphenylphosphine (1.15 g, 4.4 mmol) in THF at room temperature, diethyl azodicarboxylate (0.67 mL, 4.4 mmol) was added dropwise. The mixture was stirred at room temperature overnight, diluted with ethyl acetate and washed successively with dilute aqueous sodium hydroxide, dilute aqueous hydrochloric acid, brine, and dried over sodium sulfate. The solvents were then stripped off in vacuo. The resulting oil was washed with diethyl ether and the precipitate was filtered off. The mother liquor was adsorbed onto silica gel and then product purified by flash column chromatography (20% dichloromethane/hexanes), affording 0.12 g of a pink oil (36% yield).

¹H NMR (CDCl₃): δ 7.51 (t, J=7.5 Hz, 1H), 6.71 (d, J=9 Hz, 1H), 6.67 (d, J=10 Hz, 1H), 4.78 (q, J=7 Hz, 1H), 3.77 (s, 3H), 1.64 (d, J=7 Hz, 3H).

### Preparation 3

### (R)-2-(4-Aminomethyl-3-fluoro-phenoxy)-propionic acid methyl ester hydrochloride (IV)

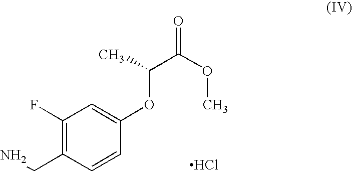

The title compound of Preparation 2 (6.5 g, 29 mmol) and palladium hydroxide (900 mg) were combined with 2.5 mL of concentrated hydrochloric acid in 200 mL of methanol and hydrogenated for 18 hr. The mixture was filtered through diatomaceous earth, concentrated by azeotropic distillation with ethanol (1×100 mL), and concentrated in vacuo to a solid. The product was suspended in 100 mL of diethyl ether, filtered, and dried to afford 7.5 g (98% yield) of the title compound.

¹H NMR (CDCl₃): δ 7.41 (t, J=8 Hz, 1H), 6.90 (br, 2H), 6.58 (m, 2H), 4.69 (q, J=7 Hz, 1H), 4.00 (s, 2H), 3.71 (s, 3H), 1.56 (d, J=7 Hz, 3H).

### Preparation 4

### (R)-2-[4-({[2-(Benzo[1,3]dioxol-5-yloxy)-pyridine-3-carbonyl]-amino}-methyl)-3-fluoro-phenoxyl-propionic acid methyl ester (II)

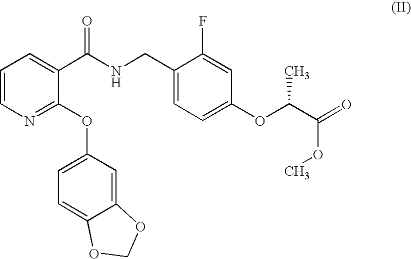

The title compound of Preparation 1 (221.9 g, 0.857 mol), the title compound of Example 3 (226.0 g, 0.857 mol), 1-hydroxybenzotriazole (127.3 g, 0.943 mol), N-ethyl-N′-(3-dimethylaminopropyl)carbodiimide hydrochloride (181.0 g, 0.943 mol), and triethylamine (119.2 mL, 86.6 g, 0.857 mol) were combined in 14 L of dichloromethane, and the resulting mixture was stirred at room temperature overnight. The mixture was washed with water (3×4 L), filtered through diatomaceous earth, and treated with decolorizing charcoal. The mixture was dried over magnesium sulfate, filtered through diatomaceous earth, and concentrated in vacuo to a solid. The solid was suspended in 2.5 L of diethyl ether and stirred overnight at ambient temperature. The solid was collected by filtration to furnish 347.3 g (87% yield) of the title compound.

MS (M/Z): 469 (M⁺+1, 20), 455 (M⁺−14, 100).

### Preparation 5

### (R)-2-[4-({[2-(Benzo[1,3]dioxol-5-yloxy)-pyridine-3-carbonyl]-amino}-methyl)-3-fluoro-phenoxy]-propionic acid (I)

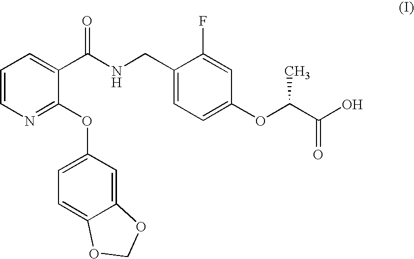

The title compound of Preparation 4 (344.0 g, 0.734 mol) was combined slowly with 1 N sodium hydroxide (1.47 L), followed by methanol (5.18 L) with ice-bath cooling. The mixture was stirred at room temperature overnight and then diluted with 3 L of water. The mixture was cooled in an ice-bath and 735 mL of 2N hydrochloric acid was added slowly dropwise. The solid was collected, dissolved in 7 L of dichloromethane, and the solution washed with brine (1×2 L). The solution was dried over sodium sulfate, filtered through diatomaceous earth, and concentrated in vacuo to a furnish solid which was recrystallized from 2.5 L of acetonitrile. There was obtained 276 g of crude product. The solid was pulped in a mixture of 2.76 L hexanes/830 mL ethyl acetate/140 mL methanol, and refluxed for 30 minutes. Upon cooling, the solid was collected, washed with hexanes, and dried to afford 253.6 g (76% yield) of the title compound, m.p. 151-152.5° C.

Anal. Calc'd. for C₂₃H₁₉FN₂O₇: C, 60.79; H, 4.21, N, 6.16. Found: C, 60.86; H, 4.35, N, 6.15.

¹H NMR (CDCl₃): δ 8.59 (dd, J=2 and 8 Hz, 1H), 8.31 (t, J=6 Hz, 1H), 8.21 (dd, J=2 and 5 Hz, 1H), 7.30 (t, J=8 Hz, 1H), 7.12 (dd, J=5and 8 Hz, 1H), 6.81 (d, J=8 Hz, 1H), 6.61 (m, 3H), 6.00 (s, 2H), 4.74 (q, J=7 Hz, 1H), 4.63 (d, J=6 Hz, 2H), 1.64 (d, J=7 Hz, 3H).

With reference to the synthetic outline depicted in Scheme 4 hereinabove, compound (la) is prepared utilizing the intermediates of the following Preparations.

### Preparation 6

### 2-(4-Fluoro-phenoxy)-nicotinic acid (VI)

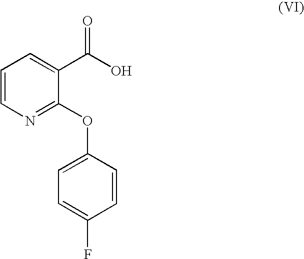

To a stirred solution of 4-fluorophenol (5.0 g, 44.6 mmole) in DMF (40 ml) at room temperature was added 60% sodium hydride (3.6 g, 89.0 mmole) portionwise and stirred for 30 min. 2-Chloronicotinic acid (7.1 g, 45.0 mmole) was added portionwise and the mixture was refluxed for three hrs. The solution was poured into 300 ml of water and washed with diethyl ether. The aqeous was poured into 400 ml of ice water and acidified to pH 3 with acetic acid. The resulting precipitate was isolated by filtration to give an off-white solid (5.2 g), m.p. 180-182° C.

MS (m/e): 234 (M⁺+1).

### Preparation 7

### 4-(1-Hydroxy-1-methyl-ethyl)-benzonitrile

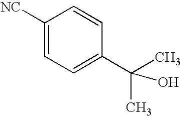

To a stirred solution of 49.5 g (0.34 mol) of 4-cyanoacetophenone in 400 mL of dry THF at −78° C. was added dropwise 150 mL (0.45 mol) of 3.0 M methyl magnesium chloride. The mixture was allowed to warm to 0° C. over three and one-half hrs, then quenched with 80 mL of methanol dropwise. The mixture was poured into 1 L of water and acidified to pH −3 with oxalic acid, then extracted with ethyl acetate (2×500 mL). The organic extracts were combined and washed with water (2×100 mL), brine (100 mL), dried over magnesium sulfate, then concentrated to give a white residue. Flash chromatography on silica gel eluting with 20% ethyl acetate/hexanes yielded 13.5 g (25%) of a clear oil that solidified on standing, m.p. 45-47° C.

### Preparation 8

### 2-(4-Aminomethyl-phenyl)-propan-2-ol

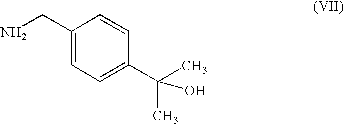

To a stirred solution of 4-(1-hydroxy-1-methyl-ethyl)-benzonitrile (20.9 g, 0.13 mol) in dry THF (300 mL) at 0° C. was added slowly dropwise 1.0 M lithium aluminum hydride in THF (388 mL, 0.39 mmol). The mixture was refluxed for 30 min., then cooled to 0° C. and quenched with methanol (50 mL) added slowly dropwise. The mixture was concentrated in vacuo to half-volume, diluted with chloroform (1200 mL), and then washed with water (300 mL). The resulting suspension was filtered through celite and the layers were separated. The organic extract was dried over magnesium sulfate and concentrated to give 16.2 g of title compound as a light yellow solid, (5.2 g), m.p. 64-66° C.

¹H NMR (CDCl₃): δ 7.45 (d, 2H), 7.26 (d, 2H), 3.83 (s, 2H), 1.57 (s, 6H).

GC-MS (m/e, %): 164 (M⁺, 15), 150 (80), 132 (75), 106 (100).

### Preparation 9

### 2-(4-Fluoro-phenoxy)-N-[4-(1-hydroxy-1-methyl-ethyl)-benzyl]-nicotinamide

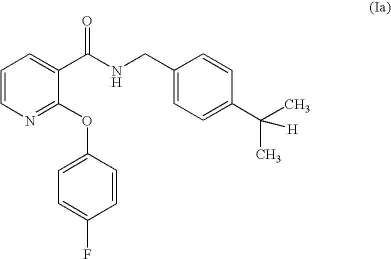

To a stirred solution of 11.3 g (48 mmol) of (VI), 8.0 g (48 mmol) of (VIl), and 7.1 g of HOBT in 200 mL of dry DMF at room temperature was added 11.0 g (57 mmol) of N-ethyl-N′-(3-dimethylaminopropyl)carbodiimide hydrochloride. The mixture was stirred at room temperature for 18 hr, and then poured into 400 mL of water and extracted with ethyl acetate (2×200 mL). The organic extracts were combined, washed with 1 N NaOH (100 mL), water (2×100 mL), brine (100 mL), dried over magnesium sulfate, and concentrated in vacuo to give a solid. Flash chromatography on silica gel eluting with 40% ethyl acetate/hexanes gave a solid. Recrystallization from ethyl acetate/hexanes (1:9) gave 16.3 g (89%) of the title compound as white crystals, m.p. 106-108° C.

Anal. Calc∝d. for C₂₂H₂₁FN₂O₃; C, 69.46; H, 5.56, N, 7.36. Found: C, 69.55; H, 5.39, N, 7.24.

### Pharmaceutical Formulations

The controlled-release formulations of the present invention, including certain preferred embodiments thereof, are set forth in detail in the following Examples.

### EXAMPLE 1

### Multiparticulate Formulations

Multiparticulate formulations comprising 50 wt % of (I), 45 wt % COMPRITOL® 888 (Gattefosse Corp., Paramus, N.J.) as a carrier, and 5 wt % PLURONIC® F127 as a dissolution enhancer are prepared by the following procedure:

First, 112.5 g of the COMPRITOL® 888,12.5 g of the PLURONIC® F127, and 2 g of water are added to a sealed, jacketed stainless-steel tank equipped with a mechanical mixing paddle. Heating fluid at 97° C. is circulated through the tank jacket. After about 40 minutes, or when a temperature of about 95° C. is reached, the mixture is molten. The mixture is then mixed at 370 rpm for 15 minutes. Next, 125 g of (I), which has been pre-heated to 95° C., is added to the melt and mixed at a speed of 370 rpm for five minutes, resulting in a feed suspension of (I) in the molten components.

Using a gear pump, the feed suspension is then pumped at a rate of 250 g/min. to the center of a four-inch diameter spinning-disk atomizer rotating at 7,500 rpm, the surface of which is maintained at about 100° C. The particles formed by the spinning-disk atomizer are congealed in ambient air and collected. The preferred average particle size is between 100 and 300 microns.

### EXAMPLE 2

Multiparticulate formulations comprising 50 wt % of (Ia), 45 wt % carnauba wax as a carrier, and 5 wt % PLURONIC® F127 or PLURONIC® F87 as a dissolution enhancer are prepared by the following melt-spray-congeal procedure:

First 112.5 g of carnauba wax and 12.5 g of the PLURONIC® F127 or PLURONIC® F87 were melted in a vessel at a temperature of about 93° C. Next, 125 of (Ia), that had been pre-heated to 95° C., was added to the melt and mixed at a speed of 370 rpm for five minutes, resulting in a feed suspension of (Ia) in the molten components.

Using a gear pump, the feed suspension was then pumped at a rate of 250 g/min. to the center of a four-inch diameter spinning-disk atomizer rotating at 5,000 rpm, the surface of which was maintained at about 100° C. The particles formed by the spinning-disk atomizer were congealed in ambient air and collected. The preferred average particle size was between 100 and 300 microns.

### EXAMPLE 3

### Short Duration Matrix Tablet (HPMC Based)

Tablet formulations of the present invention comprising 10 mg of (I) were prepared as follows.

### Drug-Containing Composition

A blend of (I), HPMC (Methocel® K100 LV; Dow Chemical Co.; Midland, Mich.) and lactose (Fast-Flo® 316; Foremost Farms USA; Baraboo, Wis.) was prepared by first passing the lactose and HPMC through a 30 mesh screen. The materials were then mixed in a Turbula® blender (GlenMills; Clifton, N.J.) for 20 minutes until a homogenous blend was achieved. The blend was then lubricated with magnesium stearate for 5 minutes in the blender. The formulations for the 10 mg dosage formulations of (I), HPMC matrix tablet short duration, are provided in Table 1.

TABLE 1
10 mg Matrix Tablet (Short Duration)
Drug-Containing Composition

| | | | Batch | |
|---|---|---|---|---|
| # | Ingredient | Purpose | (%) | mg/tab |
| 1 | (I) | PDE4D Inhib. | 2.0% | 10.0 |
| 2 | Methocel ® K100LV CR | Matrix Polymer | 35.0% | 175.0 |
| 3 | Lactose Fast-Flo ® 316 | Diluent | 62.0% | 310.0 |
| 4 | Magnesium Stearate | Lubricant | 1.0% | 5.0 |
| | | Total | 100.0% | 500.0 |

### Core

The drug-containing composition was compressed into tablet cores on an F3 tablet press (BWI Manesty; Liverpool, England) by compressing 500 mg of the drug blend for the 10 mg formulation of (I) using 7/16″ Standard Round Concave (SRC) plain faced tooling.

### EXAMPLE 4

### Long Duration Matrix Tablet (HPMC Based)

Tablet formulations of the present invention comprising of 10 mg of (I) were prepared as follows.

### Drug-Containing Composition

A blend of (I), HPMC (Methocel® K4M CR; Dow Chemical Co.; Midland, Mich.) and lactose (Fast-Flo® 316) was prepared by first passing the lactose and HPMC through a 30 mesh screen. All materials were then mixed in a Turbula® blender for 20 minutes until a homogenous blend was achieved. The blend was then lubricated with magnesium stearate for 5 minutes in the blender. The formulations for the 10 mg dosage formulations of (I), HPMC matrix tablet long duration prepared in a manner similar to that for the short duration dosages, are provided in Table 2.

TABLE 2
10 mg Matrix Tablet (Long Duration)
Drug-Containing Composition

| # | Ingredient | Purpose | Batch (%) | mg/tab |
|---|---|---|---|---|
| 1 | (I) | PDE4D Inhib. | 2.0% | 10.0 |
| 2 | Methocel ® K4M CR | Matrix Polymer | 35.0% | 175.0 |
| 3 | Lactose Fast-Flo ® 316 | Diluent | 62.0% | 310.0 |
| 4 | Magnesium Stearate | Lubricant | 1.0% | 5.0 |
| | | Total | 100.0% | 500.0 |

### Core

The drug-containing composition was compressed into tablet cores on an F3 tablet press by compressing 500 mg of the drug-containing composition for the 10 mg formulation of compound (I) using 7/16″ SRC plain faced tooling.

### EXAMPLE 5

### Short Duration Matrix Tablet (PEO Based)

Short duration matrix tablet formulations of the present invention comprising of 10 and 25 mg of (I) were prepared as follows.

### Drug-Containing Composition

A blend of compound (I), PEO (Polyox® WSR N80; Dow Chemical Co.; Midland, Mich.), lactose (Fast-Flo® 316), and HPC (Klucel® EF; Aqualon; Wilmington, Del.) was mixed in a V-blender (Patterson-Kelly; East Stroudsburg, Pa.) for 30 minutes until a homogenous blend was achieved. The blend was then granulated in an SP1 high shear mixer (Niro, Aeromatic Div.; Columbia, Md.), passed through a 12 mesh screen, and dried overnight in a tray dryer. The dried granulation was milled in a Fitzpatrick M5A mill (Fitzpatrick; Elmhurst, Ill.), and lubricated with magnesium stearate in the V-blender for 5 minutes. The formulations for the short duration 10 mg and 25 mg dosage formulations of (I) are provided in Tables 3 and 4.

TABLE 3
10 mg Matrix Tablet (Short Duration)
Drug-Containing Composition

| # | Ingredient | Purpose | Batch (%) | mg/tab |
|---|---|---|---|---|
| 1 | (I) | PDE4D Inhib. | 5.0% | 10.0 |
| 2 | Polyox ® WSR N80 | Matrix Polymer | 66.0% | 132.0 |
| 3 | Lactose Fast-Flo ® 316 | Diluent | 23.0% | 46.0 |
| 4 | HPC (Klucel ® EF) | Binder | 5.0% | 10.0 |
| 5 | Magnesium Stearate | Lubricant | 1.0% | 2.0 |
| | | Total | 100.0% | 200.0 |

TABLE 4
25 mg Matrix Tablet (Short Duration)
Drug-Containing Composition

| # | Ingredient | Purpose | Batch (%) | mg/tab |
|---|---|---|---|---|
| 1 | (I) | PDE4D Inhib. | 5.0% | 25.0 |
| 2 | Polyox ® WSR N80 | Matrix Polymer | 66.0% | 330.0 |
| 3 | Lactose Fast-Flo ® 316 | Diluent | 23.0% | 115.0 |
| 4 | HPC (Klucel ® EF) | Binder | 5.0% | 25.0 |
| 5 | Magnesium Stearate | Lubricant | 1.0% | 5.0 |
| | | Total | 100.0% | 500.0 |

### Core

The drug-containing composition was compressed into tablet cores on an F3 tablet press by compressing 200 mg of the drug-containing composition for the 10 mg formulation of (I) using 11/32″ SRC plain faced tooling. The 25 mg formulation of compound (I) was prepared by compressing 500 mg of blend using 7/16″ SRC plain faced tooling.

### In Vitro Performance of Compound (I) Formulations

A salient attribute of the matrix controlled-release formulations of the invention is the deliverty of the PDE4D inhibitor, or the pharmaceutically acceptable salt thereof, to an environment of use in a controlled manner. In vitro dissolution tests, which are well-known to one of ordinary skill in the art, may be employed to determine whether a dosage formulation provides a controlled-release profile. One example of such a dissolution test is disclosed hereinbelow. Additional examples of dissolution tests are disclosed in the aforementioned WO 01/47500.

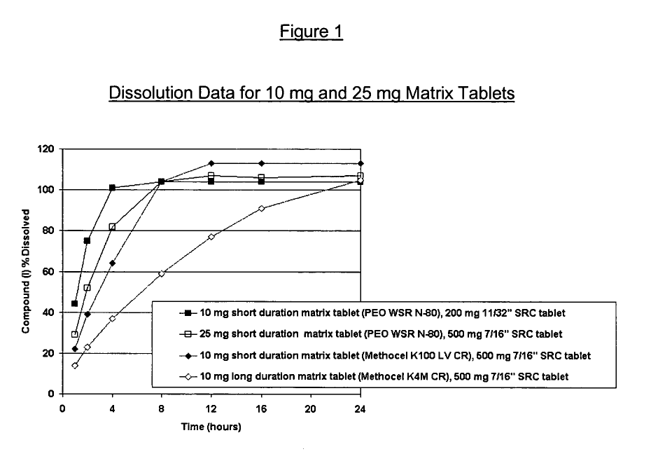

Dissolution of the 10 mg and 25 mg matrix controlled-release formulations comprising (I) was determined using an USP Apparatus II (Hansen Research Corp.; Chatsworth, Ga. or Distek Inc.; North Brunswick, N.J.) (rotating paddles at 50 rpm in 500 mL of 10 mM K₂PO₄ pH 6.8 buffer). The amount of (I) dissolved was determined by reversed-phase HPLC. The dissolution profiles exhibited release of about 100% of (I) dissolved in 4 to 24 hours depending on formulation. See FIG. 1 for the average release rate of the 10 and 25 mg formulations of HPMC and PEO based matrix tablets. The results indicate that the Polyox® WSR N80 based matrix formulations release faster than the Methocel® (HPMC) based formulations. The release rate is also faster when smaller tablets are made as can be seen for the PEO containing tablets.

### EXAMPLE 6

### Extruding Core System (ECS) Tablet Formulation

Tablet formulations of the present invention comprising 10 mg of (I) were prepared as follows.

### Drug-Containing Composition

A blend of (I), sorbitol (Neosorb® P110; Roquette America Inc.; Keokuk, Iowa), HEC (Natrosol® 250H; Aqualon; Wilmington, Del.) was mixed in a Turbula® blender for 20 minutes until a homogenous blend was achieved. The blend was then lubricated with magnesium stearate in the blender for 5 minutes.

The formulations for the drug-containing compositions for the controlled-release ECS 10 mg dosage formulations of (I) are provided in Table 5.

TABLE 5
10 mg ECS Tablet Drug-Containing Composition

| # | Ingredient | Purpose | Batch (%) | mg/tab |
|---|---|---|---|---|
| 1 | (I) | PDE4D Inhib. | 5.0% | 10.0 |
| 2 | Neosorb ® P110 | Osmotic agent | 84.0% | 168.0 |
| 3 | Natrosol ® 250H | Fluidizing agent | 10.0% | 20.0 |
| 4 | Magnesium Stearate | Lubricant | 1.0% | 2.0 |
| | | Total | 100.0% | 200.0 |

### Core

The drug-containing composition was compressed into tablet cores on an F3 tablet press by compressing 200 mg of the drug-containing composition for the 10 mg formulation of (I) using both 5/16″ SRC and 11/32″ SRC plain faced tooling.

### Coating

The tablet cores were coated in a Vector LDCS-20 coating pan (Vector Corp.; Marion, Iowa). The coating level was 9% w/w (18 mg) for the 200 mg ( 5/16″ tablet), and 12% w/w (24 mg) for the 200 mg ( 11/23″ tablet). The coating components are indicated in Table 6, and process conditions are listed hereinbelow Table 7. The coated tablets were placed in a tray drier at 45° C. for 16 hours to remove any residual coating solvents.

TABLE 6
Coating Formulation

| # | Ingredient | Batch (%) |
|---|---|---|
| 1 | Purified Water | 5.0% |
| 2 | PEG 3350 | 2.0% |
| 3 | Acetone | 89.0% |
| 4 | Cellulose Acetate (398-10) | 4.0% |
| | | 100.0% |

TABLE 7
Coating Process in LDCS-20 Coating Pan

| Process Parameter | Setting | |
|---|---|---|
| Equipment | LDCS-20 coating pan | |
| Batch Size | 600-1000 | g |
| Pan Speed | 20 | rpm |
| Process Air Flow | 30-40 | cfm |
| Inlet Air Temperature | 43° | C. |
| Outlet Air Temperature | 28° | C. |
| Atomization/Pattern Air | 22 | psi |
| Spray Rate | 20 | g/min |

### Delivery Port

A 0.9 mm diameter delivery port was drilled through the coating on the face of the tablet using a drill press having a 0.9 mm drill bit. Alternatively, the port can also be drilled using a laser.

### In Vitro Performance of Compound (I) Formulations

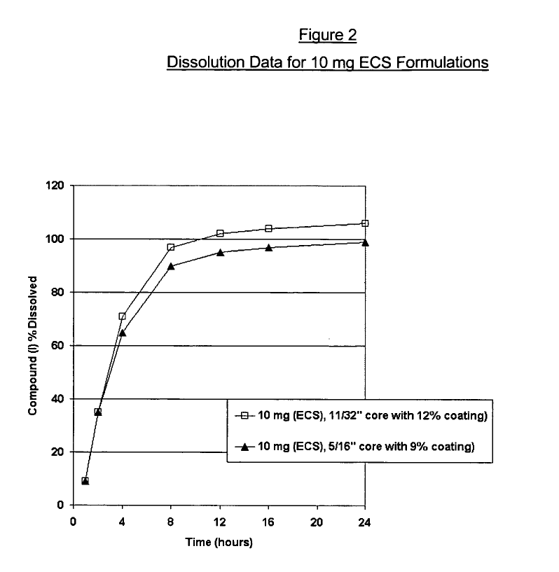

Dissolution of the 10 mg ECS controlled-release formulations comprising (I) was determined using an USP Apparatus II (rotating paddles at 50 rpm in 500 mL of 10 mM K₂PO₄ pH 6.8 buffer. The amount of (I) dissolved was determined by reversed-phase HPLC. The dissolution profile exhibited about a one-hour time lag, followed by release of greater than 90% of (I) dissolved in 12 hours. See FIG. 2 for the average release rate of the 10 mg formulations tableted with 5/16″ and 11/32″ SRC tooling.

### EXAMPLE 7

### Asymmetric Membrane Technology (AMT) Tablet Formulation

AMT tablet formulations of the present invention comprising 10 mg of (I) were prepared as follows.

### Drug-Containing Composition

A blend of (I), sorbitol (Neosorb® P110) screened through 30 mesh, microcrystalline cellulose (Avicel® PH 200; FMC Corp., Philadelphia, Pa.) was mixed in a Turbula® blender for 20 minutes until a homogenous blend was achieved. The blend was then lubricated with magnesium stearate in the blender.

The formulations for the drug-containing compositions for the controlled-release AMT 10 mg dosage formulations of (I) are provided in Table 8.

TABLE 8
10 mg Controlled Release AMI Tablet (Long
Duration) Drug-Containing Composition

| # | Ingredient | Purpose | Batch (%) | mg/tab |
|---|---|---|---|---|
| 1 | (I) | PDE4D Inhib. | 5.0% | 10.0 |
| 2 | Neosorb ® P110 | Osmotic agent | 50.0% | 88.0 |
| 3 | Avicel ® PH 200 | Diluent | 44.0 | 100.0 |
| 4 | Magnesium Stearate | Lubricant | 1.0% | 2.0 |
| | | Total | 100.0% | 200.0 |

### Core

The drug-containing composition was compressed into tablet cores on an F3 tablet press by compressing 200 mg of the drug-containing composition for the 10 mg formulation of (I) using both 5/16″ SRC and 11/32″ SRC plain faced tooling.

### Coating

The tablet cores were coated in a Vector LDCS-20 coating pan. The coating level was 9.5% w/w (19 mg) for the 200 mg ( 5/16″ tablet), and 15.2% w/w (30 mg) for the 200 mg ( 11/23″ tablet). The coating components are indicated in Table 9, and process conditions are listed hereinbelow Table 10. The coated tablets were placed in a tray drier at 45° C. for 16 hours to remove any residual coating solvents.

TABLE 9
Coating Formulation

| # | Ingredient | Batch (%) |
|---|---|---|
| 1 | Purified Water | 23.0% |
| 2 | PEG 3350 | 2.0% |
| 3 | Acetone | 67.0% |
| 4 | Cellulose Acetate (398-10) | 8.0% |
| | | 100.0% |

TABLE 10
Coating Process in LDCS-20 Coating Pan

| Process Parameter | Setting | |
|---|---|---|
| Equipment | LDCS-20 coating pan | |
| Batch Size | 600-1000 | g |
| Pan Speed | 20 | rpm |
| Process Air Flow | 30-40 | cfm |
| Inlet Air Temperature | 48° | C. |
| Outlet Air Temperature | 30° | C. |
| Atomization/Pattern Air | 22 | psi |
| Spray Rate | 20 | g/min |

### In Vitro Performance of Compound (I) Formulations

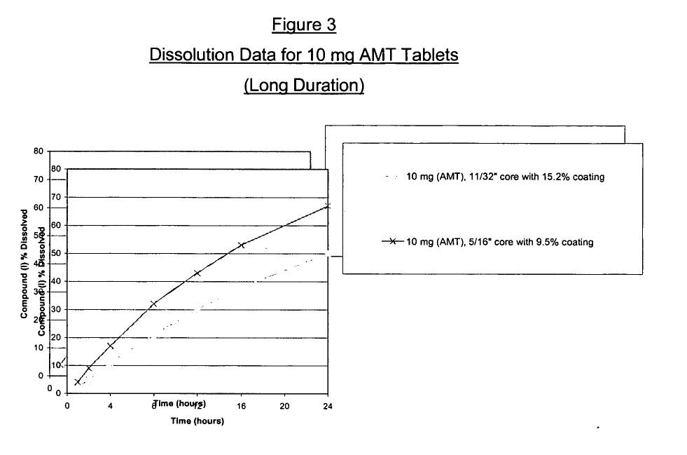

Dissolution of the 10 mg AMT controlled-release formulations comprising (I) was determined using an USP Apparatus II (rotating paddles at 50 rpm in 500 mL of 10 mM K₂PO₄ pH6.8 buffer. The amount of (I) dissolved was determined by reversed-phase HPLC. The dissolution profile exhibited about a one-hour time lag, followed by release of about 50% of (I) dissolved in 24 hours for the 11/32″ core with 15.2% AMT coating and about 67% for the 5/16″ SRC core with 9.5% AMT coating. See FIG. 3 for the average release rate of the 10 mg formulations tableted with 5/16″ and 11/32″ SRC tooling.

### EXAMPLE 8

### Short Duration Matrix Tablet (HPMC Based)

Tablet formulations of the present invention comprising 5 mg of (Ia) were prepared as follows. Two lots of (Ia) with different particle sizes were used to show the effect of drug particle size on release. The first (Ia) lot was screened and had a volume mean diameter of 182 microns. The second (la) lot was jet-milled and had a volume mean diameter of 70 microns.

### Drug-Containing Composition

A blend of (Ia), HPMC (Methocel® K100 LV) and lactose (Fast-Flo® 316) was prepared by first passing the lactose and HPMC through a 20 mesh screen. All materials were then mixed in a Turbula® blender for 20 minutes until a homogenous blend was achieved. The blend was then lubricated with magnesium stearate for 5 minutes in the blender. The formulations for the 5 mg dosage formulations of (Ia), HPMC matrix tablet short duration, are provided in Tables 11 and 12.

TABLE 11
5 mg Matrix Tablet (Short Duration)
Drug-Containing Composition with Screened Compound (Ia)

| | | | Batch | |
|---|---|---|---|---|
| # | Ingredient | Purpose | (%) | mg/tab |
| 1 | (Ia) | PDE4D Inhib. | 1.0% | 5.0 |
| 2 | Methocel ® K100LV CR | Matrix Polymer | 35.0% | 175.0 |
| 3 | Lactose Fast-Flo ® | Diluent | 63.0% | 315.0 |
| 4 | Magnesium Stearate | Lubricant | 1.0% | 5.0 |
| | | Total | 100.0% | 500.0 |

TABLE 12
5 mg Matrix Tablet (Short Duration)
Drug-Containing Composition with Jet Milled Compound (Ia)

| | | | Batch | |
|---|---|---|---|---|
| # | Ingredient | Purpose | (%) | mg/tab |
| 1 | (Ia) | PDE4D Inhib. | 1.0% | 5.0 |
| 2 | Methocel ® K100LV CR | Matrix Polymer | 35.0% | 175.0 |
| 3 | Lactose Fast-Flo ® | Diluent | 63.0% | 315.0 |
| 4 | Magnesium Stearate | Lubricant | 1.0% | 5.0 |
| | | Total | 100.0% | 500.0 |

### Core

The drug-containing compositions were compressed into tablet cores on an F3 tablet press (BWI Manesty; Liverpool, England) by compressing 500 mg of the drug blend for the 5 mg formulation of (Ia) using 7/16″ SRC plain faced tooling.

### EXAMPLE 9

### Long Duration Matrix Tablet (HPMC Based)

Tablet formulations of the invention comprising of 5 mg (Ia) were prepared as follows. Two lots of (Ia) with different particle sizes were used to show the effect of drug particle size on release. The first (Ia) lot was screened and had a volume mean diameter of 182 microns. The second (Ia) lot was jet-milled and had a volume mean diameter of 70 microns.

### Drug-Containing Composition

A blend of (Ia), HPMC (Methocel® K4M CR) and lactose (Fast-Flo® 316) was prepared by first passing the lactose and HPMC through a 20 mesh screen. All materials were then mixed in a Turbula® blender for 20 minutes until a homogenous blend was achieved. The blend was then lubricated with magnesium stearate for 5 minutes in the blender. The formulations for the 5 mg dosage formulations of (Ia), HPMC matrix tablet long duration prepared in a manner similar to that for the short duration dosages, are provided in Tables 13 and 14.

TABLE 13
5 mg Matrix Tablet (Long Duration)
Drug-Containing Composition with Screened Compound (Ia)

| # | Ingredient | Purpose | Batch (%) | mg/tab |
|---|---|---|---|---|
| 1 | (Ia) | PDE4D Inhib. | 1.0% | 5.0 |
| 2 | Methocel ® K4M CR | Matrix Polymer | 35.0% | 175.0 |
| 3 | Lactose Fast-Flo ® | Diluent | 63.0% | 315.0 |
| 4 | Magnesium Stearate | Lubricant | 1.0% | 5.0 |
| | | Total | 100.0% | 500.0 |

TABLE 14
5 mg Matrix Tablet (Long Duration)
Drug-Containing Composition with Jet Milled Compound (Ia)

| | | | Batch | |
|---|---|---|---|---|
| # | Ingredient | Purpose | (%) | mg/tab |
| 1 | (Ia) | PDE4D Inhib. | 1.0% | 5.0 |
| 2 | Methocel ® K4M CR | Matrix Polymer | 25.0% | 125.0 |
| 3 | Lactose Fast-Flo ® | Diluent | 73.0% | 365.0 |
| 4 | Magnesium Stearate | Lubricant | 1.0% | 5.0 |
| | | Total | 100.0% | 500.0 |

### Core

The drug-containing compositions were compressed into tablets on an F3 tablet press (BWI Manesty; Liverpool, England) by compressing 500 mg of the drug-containing composition for the 5 mg formulation of (Ia) using 7/16″ SRC plain faced tooling.

### In Vitro Performance of Compound (Ia) Formulations

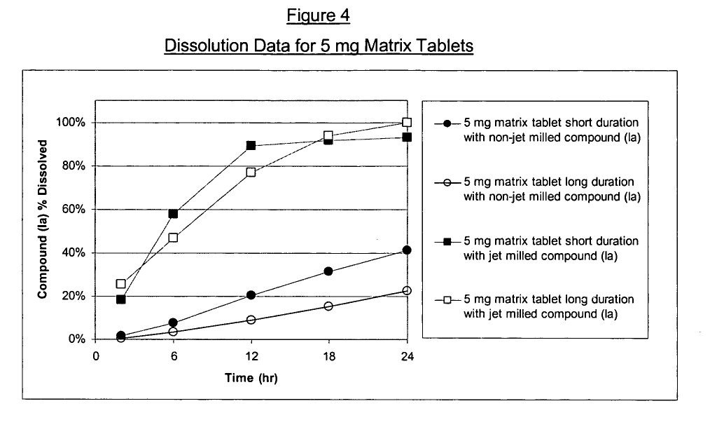

Dissolution of the 5 mg matrix controlled-release formulations comprising (Ia) was determined using an USP Apparatus II (rotating paddles at 50 rpm in 900 mL of deionized water). The amount of (Ia) dissolved was determined by reversed-phase HPLC. The dissolution profiles for formulations containing jet-milled (Ia) exhibited release of about 80% of (Ia) dissolved in 10 to 13 hours depending on formulation with nearly 100% in 24 hours. The dissolution profiles for formulations containing non jet-milled (Ia) exhibited slower release with a maximum of 20% (Ia) dissolved in 12 hours depending on formulation with a maximum of 40% in 24 hours. See FIG. 4 for the average release rate of the 4 formulations of HPMC based matrix tablets. The results indicate that the higher molecular weight polymer (Methocel® K4M) may initially release faster than the lower molecular weight K100 LV CR. After the polymer gels more completely (after 2 hours) the trend is reversed as would be expected. Formulations made with jet-milled (I) exhibited much faster and more complete release within a 24-hour period.

### EXAMPLE 10

### ECS Tablet Formulations

Tablet formulations of the present invention comprising of 5 mg of (Ia) were prepared as follows. Two lots of (Ia) with different particle sizes were used to show the effect of drug particle size on release. The first (Ia) lot was screened and had a volume mean diameter of 182 microns. The second (Ia) lot was jet-milled and had a volume mean diameter of 70 microns.

### Drug-Containing Composition

A blend of (Ia), sorbitol (Neosorb® P110), and HEC (Natrosol® 250H) was mixed in a Turbula® blender for 20 minutes until a homogenous blend was achieved. The blend was then lubricated with magnesium stearate in the blender for 5 minutes.

The formulations for the drug-containing compositions for the controlled-release ECS 5 mg dosage formulations of (Ia) are provided in Tables 15 and 16.

TABLE 15
5 mg ECS Tablet
Drug-Containing Composition with Screened Compound (Ia)

| # | Ingredient | Purpose | Batch (%) | mg/tab |
|---|---|---|---|---|
| 1 | (Ia) | PDE4D Inhib. | 2.5% | 5.0 |
| 2 | Neosorb ® P110 | Osmotic agent | 86.4% | 172.8 |
| 3 | Natrosol ® 250H | Fluidizing agent | 10.0% | 20.0 |
| 4 | FD & C Blue Lake #2 | Colorant | 0.1% | 0.2 |
| 5 | Magnesium Stearate | Lubricant | 1.0% | 2.0 |
| | | Total | 100.0% | 200.0 |

TABLE 16
5 mg ECS Tablet
Drug-Containing Composition with Jet Milled Compound (Ia)

| # | Ingredient | Purpose | Batch (%) | mg/tab |
|---|---|---|---|---|
| 1 | (Ia) | PDE4D Inhib. | 2.5% | 5.0 |
| 2 | Neosorb ® P110 | Osmotic agent | 86.4% | 172.8 |
| 3 | Natrosol ® 250H | Fluidizing agent | 10.0% | 20.0 |
| 4 | FD & C Blue Lake #2 | Colorant | 0.1% | 0.2 |
| 5 | Magnesium Stearate | Lubricant | 1.0% | 2.0 |
| | | Total | 100.0% | 200.0 |

### Core

The drug-containing compositions were compressed into tablet cores on an F3 tablet press (BWI Manesty, Liverpool, England) by compressing 200 mg of the drug-containing composition for the 5 mg formulation of (Ia) using both 11/32″ SRC plain faced tooling and 0.1969″ wide×0.3937″ long modified oval tooling.

### Coating

The tablet cores were coated in a Vector LDCS-20 coating pan. The coating level for formulations containing jet-milled (Ia) was 11.0% w/w (22 mg) for the 200 mg ( 11/32″ SRC tablet), and 12.0% w/w (24 mg) for the 200 mg (oval tablet). The coating level for formulations containing non jet-milled (Ia) was 14.0% w/w (28 mg) for the 200 mg ( 11/32″ SRC tablet), and 9.0% w/w (18 mg) for the 200 mg ( 11/32″ tablet). The coating components are indicated in Table 17, and process conditions are listed hereinbelow Table 18. The coated tablets were placed in a tray drier at 45° C. for 16 hours to remove any residual coating solvents.

TABLE 17
Coating Formulation

| # | Ingredient | Batch (%) |
|---|---|---|
| 1 | Purified Water | 5.0% |
| 2 | PEG 3350 | 2.0% |
| 3 | Acetone | 89.0% |
| 4 | Cellulose Acetate (398-10) | 4.0% |
| | | 100.0% |

TABLE 18
Coating Process in LDCS-20 Coating Pan

| Process Parameter | Setting | |
|---|---|---|
| Equipment | LDCS-20 coating pan | |
| Batch Size | 600-1000 | g |
| Pan Speed | 20 | rpm |
| Process Air Flow | 30-40 | cfm |
| Inlet Air Temperature | 43° | C. |
| Outlet Air Temperature | 28° | C. |
| Atomization/Pattern Air | 22 | psi |
| Spray Rate | 20 | g/min |

### Delivery Port

A 0.9 mm diameter delivery port was drilled through the coating on the face of the tablet for the 11/32″ SRC tablets and through the band for the oval tablets using a drill press having a 0.9 mm drill bit. The port can also be drilled using a laser.

### In Vitro Performance of Compound (Ia) Formulations

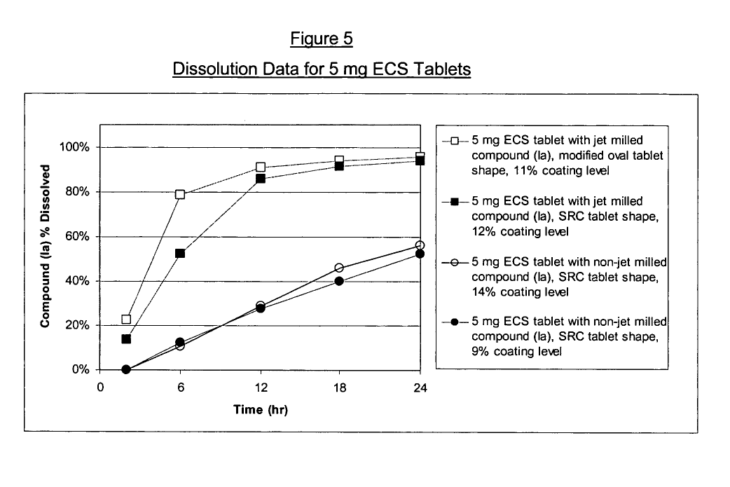

Dissolution of the 5 mg ECS controlled-release formulations comprising (Ia) was determined using an USP Apparatus II (rotating paddles at 50 rpm in 900 mL of deionized water). The amount of (Ia) dissolved was determined by reversed-phase HPLC. The dissolution profiles of formulations containing jet-milled (Ia) exhibited a time lag, followed by release of than 80% of (Ia) in 7 hours for the oval tablet and 11 hours for the SRC tablet configuration. The dissolution profiles of formulations containing non jet-milled (I) exhibited a time lag and did not reach 80% of (Ia) in 24 hours for the SRC tablet shape. See FIG. 5 for the average release rate of the 5 mg formulations tabletted with 11/32″ SRC and modified oval tooling. The formulations with the jet-milled (Ia) showed much faster and more complete release within 24 hours compared to the formulations that were not jet-milled. The modified oval tooling showed a faster release rate compared to the SRC shape. Release was independent of the amount of coating over the 11 to 14% range.

1. A controlled-release pharmaceutical formulation comprising a phosphodiesterase type 4D (PDE4D) inhibitor, or a pharmaceutically acceptable salt thereof, said formulation exhibiting at least one of the following characteristics:
(i) a Tₘₐₓ of greater than about 1.5 hours;
(ii) less than about 80% of said PDE4D inhibitor, or said pharmaceutically acceptable salt thereof, is released in vivo at about 1.5 hours;
(iii) less than about 80% of said PDE4D inhibitor, or said pharmaceutically acceptable salt thereof, is released in vitro at about 1.5 hours; or
(iv) an in vivo delivery lag time prior to initiation of release of said PDE4D inhibitor, or said pharmaceutically acceptable salt thereof, of between about 0.5 hours and about four hours, wherein said formulation comprises an asymmetric membrane-coated osmotic tablet, capsule, or bead core comprising:
(a) a PDE4D inhibitor, or a pharmaceutically acceptable salt thereof;
(b) one or more osmotic agents; and
(c) at least one asymmetric membrane coating said tablet, capsule, or bead core.

2. A formulation of claim 1, wherein said PDE4D inhibitor is [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof, or 2-(4-fluoro-phenoxy)-N-[4-(1-hydroxy-1-methyl-ethyl)-benzyl]-nicotinamide, or a pharmaceutically acceptable salt thereof.

3. A formulation of claim 1, wherein said osmotic agent is lactose, mannitol, sorbitol, or sodium bitartrate.

4. A formulation of claim 1, further comprising a solubilizing agent

5. A formulation of claim 4, wherein said solubilizing agent comprises an organic acid, an organic base, an inorganic acid, an inorganic base, a surfactant, or a cyclodextrin.

6. A formulation of claim 1, further comprising a tableting aid.

7. A formulation of claim 6, wherein said tableting aid comprises from about 20% to about 70% w/w microcrystalline cellulose and from about 0.5% to about 2% w/w magnesium stearate.

8. A formulation of claim 1, wherein said PDE4D inhibitor comprises [(R)-2-[4-({[2-benzo[1,3]dioxol-5-yloxy)-pyridine-3-carbonyl]-amino[-methyl]-3-fluoro-phenoxy]-propionic acid, or a pharmaceutically acceptable salt thereof; said osmotic agent comprises from about 25% to about 75% w/w sorbitol; said asymmetric membrane coating comprises cellulose acetate and polyethylene glycol, said coating comprising from about 3% to about 20% w/w of said core.

9. A controlled-release pharmaceutical formulation comprising a phosphodiesterase type 4D (PDE4D) inhibitor, or a pharmaceutically acceptable salt thereof, said formulation exhibiting at least one of the following characteristics:
(i) a Tₘₐₓ of greater than about 1.5 hours;
(ii) less than about 80% of said PDE4D inhibitor, or said pharmaceutically acceptable salt thereof, is released in vivo at about 1.5 hours;
(iii) less than about 80% of said PDE4D inhibitor, or said pharmaceutically acceptable salt thereof, is released in vitro at about 1.5 hours; or
(iv) an in vivo delivery lag time prior to initiation of release of said PDE4D inhibitor, or said pharmaceutically acceptable salt thereof, of between about 0.5 hours and about four hours, wherein said formulation comprises a membrane-coated osmotic tablet comprising:
(a) a PDE4D inhibitor, or a pharmaceutically acceptable salt thereof;
(b) a hydroxyethyl cellulose having a weight average molecular weight of from about 300,000 to about 2,000,000;
(c) an osmagent; and
(d) a water-permeable membrane coating said osmotic tablet, wherein said membrane coating comprises at least one delivery port therethrough.

10. A formulation of claim 9, wherein said PDE4D inhibitor is [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof, or 2-(4-fluoro-phenoxy)-N-[4-(1-hydroxy-1-methyl-ethyl)-benzyl]-nicotinamide, or a pharmaceutically acceptable salt thereof.

11. A formulation of claim 9, wherein said hydroxyethyl cellulose is present in said tablet core from about 3% to about 15% w/w.

12. A formulation of claim 9, wherein said osmagent is sorbitol and is present in said tablet core from about 50% to about 90% w/w.

13. A formulation of claim 9, wherein said water permeable membrane coating comprises cellulose acetate and polyethylene glycol, said coating comprising from about 5% to about 15% w/w of said core.

14. A controlled-release pharmaceutical formulation comprising a phosphodiesterase type 4D (PDE4D) inhibitor, or a pharmaceutically acceptable salt thereof, said formulation exhibiting at least one of the following characteristics:
(i) a Tₘₐₓ of greater than about 1.5 hours;
(ii) less than about 80% of said PDE4D inhibitor, or said pharmaceutically acceptable salt thereof, is released in vivo at about 1.5 hours;
(iii) less than about 80% of said PDE4D inhibitor, or said pharmaceutically acceptable salt thereof, is released in vitro at about 1.5 hours; or
(iv) an in vivo delivery lag time prior to initiation of release of said PDE4D inhibitor, or said pharmaceutically acceptable salt thereof, of between about 0.5 hours and about four hours, wherein said formulation comprises:
(a) a PDE4D inhibitor, or a pharmaceutically acceptable salt thereof; and
(b) a polymer that moderates release of said PDE4D inhibitor, or said pharmaceutically acceptable salt thereof.

15. A formulation of claim 14, wherein said PDE4D inhibitor is [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof, or 2-(4-fluoro-phenoxy)-N-[4-(1-hydroxy-1-methyl-ethyl)-benzyl]-nicotinamide, or a pharmaceutically acceptable salt thereof.

16. A formulation of claim 14, wherein said polymer comprises a hydrophilic polymer that swells, thereby forming a gel, in the presence of water.

17. A formulation of claim 16, wherein said hydrophilic polymer is hydroxypropylmethyl cellulose, polyethylene oxide, polyacrylic acid, polyvinyl alcohol, hydroxypropyl cellulose, methyl cellulose, carboxymethyl cellulose sodium, polyvinylpyrrolidone, or poly(2-hydroxyethyl methacrylate).

18. A formulation of claim 14, further comprising a diluent, a solubilizing agent, or a tableting aid.

19. A formulation of claim 14, wherein said PDE4D inhibitor comprises [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof; said polymer comprises hydroxypropylmethyl cellulose having a methoxyl content from about 19% to about 24% w/w and a hydroxypropoxyl content from about 7% to about 12% w/w and present in said formulation at from about 15% to about 55% w/w; and a diluent comprising lactose present in said formulation at from about 43% to about 83% w/w of said formulation.

20. A formulation of claim 14, wherein said PDE4D inhibitor comprises 2-(4-fluoro-phenoxy)-N-[4-(1-hydroxy-1-methyl-ethyl)-benzyl]-nicotinamide, or a pharmaceutically acceptable salt thereof; said polymer comprises hydroxypropylmethyl cellulose having a methoxyl content from about 19% to about 24% w/w and a hydroxypropoxyl content from about 7% to about 12% w/w and present in said formulation at from about 15% to about 55% w/w; and a diluent comprising lactose present in said formulation at from about 43% to about 83% w/w of said formulation.

21. A formulation of claim 14, wherein said polymer is a hydrophobic polymer selected from the group consisting of fatty acids, fatty alcohols, and fatty acid esters.

22. A formulation of claim 14, wherein said polymer comprises a pharmaceutically inert polymer selected from the group consisting of polyvinyl chloride, polyvinyl acetate, methylacrylate, or methymethacrylate, and co-polymers thereof.

23. A controlled-release pharmaceutical formulation comprising a phosphodiesterase type 4D (PDE4D) inhibitor, or a pharmaceutically acceptable salt thereof, said formulation exhibiting at least one of the following characteristics:
(i) a Tₘₐₓ of greater than about 1.5 hours;
(ii) less than about 80% of said PDE4D inhibitor, or said pharmaceutically acceptable salt thereof, is released in vivo at about 1.5 hours;
(iii) less than about 80% of said PDE4D inhibitor, or said pharmaceutically acceptable salt thereof, is released in vitro at about 1.5 hours; or
(iv) an in vivo delivery lag time prior to initiation of release of said PDE4D inhibitor, or said pharmaceutically acceptable salt thereof, of between about 0.5 hours and about four hours, wherein said formulation comprises:
(a) a PDE4D inhibitor, or a pharmaceutically acceptable salt thereof;
(b) a carrier; and
(c) a dissolution-enhancing agent.

24. A formulation of claim 23, wherein said PDE4D inhibitor is [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof, or 2-(4-fluoro-phenoxy)-N-[4-(1-hydroxy-1-methyl-ethyl)-benzyl]-nicotinamide, or a pharmaceutically acceptable salt thereof.

25. A formulation of claim 23, wherein said dissolution-enhancing agent is an emulsifying agent, a sugar, an alcohol, a salt, an amino acid, or a mixture thereof.

26. A formulation of claim 23, wherein said dissolution-enhancing agent is a poloxamer, a polyoxyethylene alkyl ester or ether, a polyoxyethylene castor oil derivative, or a polyoxyethylene sorbitan fatty acid ester.

27. A formulation of claim 23, wherein said carrier is a wax, a glyceride, or a mixture thereof, wherein said carrier is different from said dissolution-enhancing agent.

28. A formulation of claim 27, wherein said carrier is a synthetic wax, a microcrystalline wax, a paraffin wax, carnauba wax, glyceryl monooleate, glyceryl monostearate, glyceryl palmitostearate, a polyethoxylated castor oil derivative, a hydrogenated vegetable oil, a glyceryl mono-, di-, or tri-behenate, glyceryl tristearate, glyceryl tripalmitate, or a mixture thereof, wherein said carrier is different from said dissolution enhancing agent.

29. A formulation of claim 23, wherein said formulation comprises from about 20% to about 75% w/w of [(R)-2-[4-({[2-benzo[1,3]dioxol-5-yloxy)-pyridine-3-carbonyl]-amino}-methyl]-3-fluoro-phenoxy]-propionic acid, or a pharmaceutically acceptable salt thereof, from about 25% to about 80% w/w of said carrier, and from about 0.1% to about 30% w/w of said dissolution-enhancing agent.

30. A formulation of claim 23, wherein said formulation comprises from about 20% to about 75% w/w of 2-(4-fluoro-phenoxy)-N-[4-(1-hydroxy-1-methyl-ethyl)-benzyl]-nicotinamide, or a pharmaceutically acceptable salt thereof, from about 25% to about 80% w/w of said carrier, and from about 0.1% to about 30% w/w of said dissolution-enhancing agent.

31. A method of treating disorders and conditions mediated by the PDE4D isozyme, which method comprises administering to a mammal in need of such treatment a therapeutically effective amount of a PDE4D inhibitor, or a pharmaceutically acceptable salt thereof, in a controlled-release formulation of any one of claims 1, 9, 14, or 23.

32. A method of claim 31, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.

33. A method of claim 31, wherein said disorder or condition is:
(a) an inflammatory disease or condition selected from the group consisting of joint inflammation, rheumatoid arthritis, rheumatoid spondylitis, osteoarthritis, inflammatory bowel disease, ulcerative colitis, chronic glomerulonephritis, dermatitis, and Crohn's Disease;
(b) a respiratory disease or condition selected from the group consisting of asthma, acute respiratory distress syndrome, chronic obstructive pulmonary disease, bronchitis, chronic obstructive airway disease, and silicosis;
(c) an infectious disease or condition selected from the group consisting of sepsis, septic shock, endotoxic shock, gram negative sepsis, toxic shock syndrome, fever and myalgias due to bacterial, viral or fungal infection, and influenza;
(d) an immune disease or condition selected from the group consisting of autoimmune diabetes, systemic lupus erythematosis, graft v. host reaction, allograft rejections, multiple sclerosis, psoriasis, and allergic rhinitis; or
(e) a disease or condition selected from the group consisting of bone resorption diseases, reperfusion injury, cachexia secondary to infection or malignancy, cachexia secondary to human immunodeficiency syndrome (AIDS), human immunodeficiency virus (HIV), infection, or AIDS related complex (ARC), keloid formation, scar tissue formation, Type 1 diabetes mellitus, and leukemia.

34. A method of claim 33, wherein said disorder or condition is asthma, acute respiratory distress syndrome, chronic obstructive pulmonary disease, bronchitis, chronic obstructive airway disease, or silicosis.

35. A method of reducing PDE4D inhibitor treatment-induced nausea and/or emesis in a mammal which method comprises administering to said mammal said PDE4D inhibitor, or a pharmaceutically acceptable salt thereof, in a controlled-release formulation of any one of claims 1, 9, 14, or 23.

36. A method of claim 35, wherein said PDE4D inhibitor comprises (R)-2-[4-({[2-(benzo[1,3]dioxol-5-yloxy)-pyridine-3-carbonyl]-amino}-methyl)-3-fluoro-phenoxy]-propionic acid, or a pharmaceutically acceptable salt thereof, or 2-(4-fluorophenoxy)-N-[4-(1-hydroxy-1-methyl-ethyl)-benzyl]-nicotinamide, or a pharmaceutically acceptable salt thereof.
